SEQUENCE LISTING (32 sequences)

SEQ ID NO: 1 - DNA, 37 bp, Artificial Sequence
  Description of Artificial Sequence Synthetic Synthetic primer
gcccccaagcttgccgccaccatgctgccgtgcctcg

SEQ ID NO: 2 - DNA, 34 bp, Artificial Sequence
  Description of Artificial Sequence Synthetic primer
atcgggggatccgttggtcacggtgaaatactgc

SEQ ID NO: 3 - DNA, 28 bp, Artificial Sequence
  Description of Artificial Sequence Synthetic primer
cgcggatccggtgagggagatgcactcc

SEQ ID NO: 4 - DNA, 29 bp, Artificial Sequence
  Description of Artificial Sequence Synthetic primer
cgggatcctctgacaaaactcacacatcc

SEQ ID NO: 5 - DNA, 26 bp, Artificial Sequence
  Description of Artificial Sequence Synthetic primer
cgcggatccgcacctgaactcctggg

SEQ ID NO: 6 - DNA, 32 bp, Artificial Sequence
  Description of Artificial Sequence Synthetic primer
atcggggaattctcatttacccggagacaggg

SEQ ID NO: 7 - DNA, 1398 bp, Homo sapiens
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacggatcctctgacaaaactcacacatccccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - protein, 465 aa, Homo sapiens [sequence omitted]

SEQ ID NO: 9 - DNA, 1341 bp, Homo sapiens
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccggatccgcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - protein, 446 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTGSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 3649 bp, Homo sapiens
agtcccagcccaagggtagctggaggcgcgcaggccggctccgctccggccccggacgatgcggcgcgcccaggatgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtcatcatcttctttgcctttgtcctgctgctctccggagccctcgcctactgcctggccctccagctgtatgtgcggcgccgaaagaagctacccagtgtcctgctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacggggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagtgactcgggctgagaggctgcttttgattttagccatgcctgctcctctgcctggaccaggaggagggcccctggggcagaagttaggcacgaggcagtctgggcacttttctgcaagtccactggggctggccccagccaggccctgcagggctggtcagggtgtctggggcaggaggaggccaactcactgaactagtgcagggtatgtgggtggcactgacctgttctgttgactggggccctgcagactctggcagagctgagaagggcagggaccttctccctcctaggaactctttcctgtatcataaaggattatttgctcaggggaaccatggggctttctggagttgtggtgaggccaccaggctgaagtcagctcagacccagacctccctgcttaggccactcgagcatcagagcttccagcaggaggaagggctgtaggaatggaagcttcagggccttgctgctggggtcatttttaggggaaaaaggaggatatgatggtcacatggggaacctcccctcatcgggcctctggggcaggaagcttgtcactggaagatcttaaggtatatattttctggacactcaaacacatcataatggattcactgaggggagacaaagggagccgagaccctggatggggcttccagctcagaacccatccctctggtgggtacctctggcacccatctgcaaatatctccctctctccaacaaatggagtagcatccccctggggcacttgctgaggccaagccactcacatcctcactttgctgccccaccatcttgctgacaacttccagagaagccatggttttttgtattggtcataactcagccctttgggcggcctctgggcttgggcaccagctcatgccagccccagagggtcagggttggaggcctgtgcttgtgtttgctgctaatgtccagctacagacccagaggataagccactgggcactgggctggggtccctgccttgttggtgttcagctgtgtgattttggactagccacttgtcagagggcctcaatctcccatctgtgaaataaggactccacctttaggggaccctccatgtttgctgggtattagccaagctggtcctgggagaatgcagatactgtccgtggactaccaagctggcttgtttcttatgccagaggctaacagatccaatgggagtccatggtgtcatgccaagacagtatcagacacagccccagaagggggcattatgggccctgcctccccataggccatttggactctgccttcaaacaaaggcagttcagtccacaggcatggaagctgtgaggggacaggcctgtgcgtgccatccagagtcatctcagccctgcctttctctggagcattctgaaaacagatattctggcccagggaatccagccatgacccccacccctctgccaaagtactcttaggtgccagtctggtaactgaactccctctggaggcaggcttgagggaggattcctcagggttcccttgaaagctttatttatttattttgttcatttatttattggagaggcagcattgcacagtgaaagaattctggatatctcaggagccccgaaattctagctctgactttgctgtttccagtggtatgaccttggagaagtcacttatcctcttggagcctcagtttcctcatctgcagaataatgactgacttgtctaattcgtagggatgtgaggttctgctgaggaaatgggtatgaatgtgccttgaacacaaagctctgtcaataagtgatacatgttttttattccaataaattgtcaagaccaca

SEQ ID NO: 12 - DNA, 768 bp, Homo sapiens
atgaaaaagacagctatcgcgattgcagtggcactggctggtttcgctaccgtagcgcaggccgacgtcgagtccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 13 - protein, 578 aa, Homo sapiens
  U00672
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNGEPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 14 - DNA, 3632 bp, Homo sapiens
  U00672
aaagagctggaggcgcgcaggccggctccgctccggccccggacgatgcggcgcgcccaggatgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtcatcatcttctttgcctttgtcctgctgctctccggagccctcgcctactgcctggccctccagctgtatgtgcggcgccgaaagaagctacccagtgtcctgctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacggggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagtgactcgggctgagaggctgcttttgattttagccatgcctgctcctctgcctggaccaggaggagggccctggggcagaagttaggcacgaggcagtctgggcacttttctgcaagtccactggggctggcccagccaggctgcagggctggtcagggtgtctggggcaggaggaggccaactcactgaactagtgcagggtatgtgggtggcactgacctgttctgttgactggggccctgcagactctggcagagctgagaagggcagggaccttctccctcctaggaactctttcctgtatcataaaggattatttgctcaggggaaccatggggctttctggagttgtggtgaggccaccaggctgaagtcagctcagacccagacctccctgcttaggccactcgagcatcagagcttccagcaggaggaagggctgtaggaatggaagcttcagggccttgctgctggggtcatttttaggggaaaaaggaggatatgatggtcacatggggaacctcccctcatcgggcctctggggcaggaagcttgtcactggaagatcttaaggtatatattttctggacactcaaacacatcataatggattcactgaggggagacaaagggagccgagaccctggatggggcttccagctcagaacccatccctctggtgggtacctctggcacccatctgcaaatatctccctctctccaacaaatggagtagcatccccctggggcacttgctgaggccaagccactcacatcctcactttgctgccccaccatcttgctgacaacttccagagaagccatggttttttgtattggtcataactcagccctttgggcggcctctgggcttgggcaccagctcatgccagccccagagggtcagggttggaggcctgtgcttgtgtttgctgctaatgtccagctacagacccagaggataagccactgggcactgggctggggtccctgccttgttggtgttcagctgtgtgattttggactagccacttgtcagagggcctcaatctcccatctgtgaaataaggactccacctttaggggaccctccatgtttgctgggtattagccaagctggtcctgggagaatgcagatactgtccgtggactaccaagctggcttgtttcttatgccagaggctaacagatccaatgggagtccatggtgtcatgccaagacagtatcagacacagccccagaagggggcattatgggccctgcctccccataggccatttggactctgccttcaaacaaaggcagttcagtccacaggcatggaagctgtgaggggacaggcctgtgcgtgccatccagagtcatctcagccctgcctttctctggagcattctgaaaacagatattctggcccagggaatccagccatgacccccacccctctgccaaagtactcttaggtgccagtctggtaactgaactccctctggaggcaggcttgagggaggattcctcagggttcccttgaaagctttatttatttattttgttcatttatttattggagaggcagcattgcacagtgaaagaattctggatatctcaggagccccgaaattctagctctgactttgctgtttccagtggtatgaccttggagaagtcacttatcctcttggagcctcagtttcctcatctgcagaataatgactgacttgtctaattcatagggatgtgaggttctgctgaggaaatgggtatgaatgtgccttgaacacaaagctctgtcaataagtgatacatgttttttattccaataaattgtcaagaccaca

SEQ ID NO: 15 - DNA, 708 bp, Artificial Sequence
  Description of Artificial Sequence Synthetic V12
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaactga

SEQ ID NO: 16 - protein, 235 aa, Artificial Sequence
  Description of Artificial Sequence Synthetic V12
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 17 - DNA, 1365 bp, Artificial Sequence
  Description of Artificial Sequence Synthetic V51
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacggatccgcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 454 aa, Artificial Sequence
  Description of Artificial Sequence Synthetic V51
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNGSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1410 bp, Artificial Sequence
  Description of Artificial Sequence Synthetic V52
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacggatccgagtccaaatcttctgacaaaactcacacatccccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 469 aa, Artificial Sequence
  Description of Artificial Sequence Synthetic V52
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNGSESKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1410 bp, Artificial Sequence
  Description of Artificial Sequence Synthetic V55
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacggatccgagtccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 469 aa, Artificial Sequence
  Description of Artificial Sequence Synthetic V55
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNGSESKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 37 bp, Artificial Sequence
  Description of Artificial Sequence Synthetic primer
atcggggaattctcagttggtcacggtgaaatactgc

SEQ ID NO: 24 - DNA, 29 bp, Artificial Sequence
  Description of Artificial Sequence Synthetic primer
cgggatcctctgacaaaactcacacatcc

SEQ ID NO: 25 - DNA, 30 bp, Artificial Sequence
  Description of Artificial Sequence Synthetic primer
ctcacacatccccaccgtccccagcacctg

SEQ ID NO: 26 - DNA, 31 bp, Artificial Sequence
  Description of Artificial Sequence Synthetic primer
acggtggggatgtgtgagttttgtcagaaga

SEQ ID NO: 27 - DNA, 34 bp, Artificial Sequence
  Description of Artificial Sequence Synthetic primer
cgcggatccgagtccaaatcttgtgacaaaactc

SEQ ID NO: 28 - DNA, 30 bp, Artificial Sequence
  Description of Artificial Sequence Synthetic primer
gtgacaaaactcacacatgcccaccgtgcc

SEQ ID NO: 29 - DNA, 31 bp, Artificial Sequence
  Description of Artificial Sequence Synthetic primer
atgtgtgagttttgtcacaagatttggactc

SEQ ID NO: 30 - DNA, 1410 bp, Artificial Sequence
  Description of Artificial Sequence Synthetic V54
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacggatccgagtccaaatcttgtgacaaaactcacacatccccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 31 - protein, 469 aa, Artificial Sequence
  Description of Artificial Sequence Synthetic V54
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNGSESKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 34 bp, Artificial Sequence
  Description of Artificial Sequence Synthetic primer
cgcggatccgagtccaaatcttctgacaaaactc

(19) United States
(12) Patent Application Publication
(10) Pub. No.: US 2009/0111146 A1
(43) Pub. Date: Apr. 30, 2009

(54) Antibody Drug

(75) Inventors: Eiko Ohtsuka, Sapporo-shi (JP); Yutaka Tamura, Sapporo-shi (JP)

Correspondence Address: BIRCH STEWART KOLASCH & BIRCH, PO BOX 747, FALLS CHURCH, VA, 22040-0747, US

(73) Assignee: National Institute of Advanced Industrial Science, Tokyo (JP)

(21) Appl. No.: 10/570,143

(22) PCT Filed: Sep. 2, 2004
(86) PCT No.: PCT/JP04/13090

§ 371 Date: Mar. 31, 2007

Related U.S. Application Data
(60) Provisional application No. 60/539,745, filed on Jan. 29, 2004.

(30) Foreign Application Priority Data
Sep. 2, 2003 (JP) 2003-310601

(51) Int. Cl. C12P 21/04; C12N 15/00; C07K 16/18; C12N 15/11; C12N 5/06

(52) U.S. Cl. 435/69.7; 435/320.1; 530/387.3; 536/23.4; 435/326

(57) ABSTRACT

Means for effectively performing therapies at a low cost is provided in which an antibody drug including a fusion protein fusing an extracellular region of IL-10 receptor 1 with a human antibody is used. A gene encoding a fusion protein fusing an extracellular region of IL-10 receptor 1 with a constant region of human IgG1 is incorporated into an expression vector to provide an expression vector for gene therapies and vaccines.

### TECHNICAL FIELD

The present invention relates to techniques of a drug in which an antibody is utilized, an antibody drug, and a vector for causing an antibody drug to be expressed. More specifically, the invention relates to an expression vector for expressing a gene encoding a fusion protein in which an Fc region of a human antibody is bound to a fragment of IL-10 receptor 1.

### BACKGROUND ART

[Chimeric Antibody, Humanized Antibody]

Monoclonal antibodies are highly specific, and have been expected to specifically eliminate target cells such as cancer cells. However, antibodies of animals other than human such as mouse have been prepared on the ground that a myeloma cell suited for preparation of monoclonal antibodies was not found in human.

However, heterologous animal antibodies have many parts specific for the heterologous animal, and therefore, when they are administered intact to a human as a drug, problems of occurrence of immunoreaction against the heterologous animal antibody may be involved.

Hence, preparation of chimeric antibodies has been attempted. A cDNA of a mouse immunoglobulin variable region, and a constant region (Fc region) of immunoglobulin derived from human were bound and expressed (Nonpatent Document 1: Nature. (1984) Vol. 312, P. 643-6.). However, 70% of it was regions derived from human, and thus still caused the immunoreaction.

Thereafter, Winter et al. found that three loops (CDR, complementary determining regions) in the variable region of immunoglobulin serve to bind the antibody to the antigen.

Consequently, production of an antibody all but 5-10% of which is derived from human was enabled by designing all parts other than these three loops from the variable region to be derived from human (Nonpatent Document 2: Nature (1986) Vol. 321, p. 783-792,). Moreover, a process referred to as reshape in which in this antibody design an antigen-binding site derived from a mouse antibody is grafted into a human antibody framework region was also developed (Nonpatent Document 3: Nature 1988 Vol. 332,323-). In addition, prevention of deterioration of affinity accompanying humanization was planned (Patent Document 1: U.S. Pat. No. 6,180,370). Humanized antibodies which do not substantially cause immunoreaction and have affinity were prepared, whereby the groundwork for exploiting the antibodies as a drug has been laid.

[Immunoadhesin]

On the other hand, as gene sequences of human antibodies are elucidated, attempts to cause a fragment of a target protein and the Fc region of a human antibody to be expressed as a fusion protein (immunoadhesin) have been made using a gene recombination technique.

For example, it was suggested that immunoadhesin prepared by fusing an extracellular region of TNFR (TNF receptor), and a hinge part and the Fc region of a human IgG heavy chain serves as a TNF antagonist (Nonpatent Document 4: PRONAS Vol. 88, p. 10535-19539).

[IL-10R Interleukin 10 Receptor]

IL-10 (interleukin 10) is predominantly produced by helper T cells (type 2). On one hand, IL-10 has an immunosuppressive activity to inhibit synthesis of a variety of cytokines, interferon γ, IL-2, and TNF (tumor necrosis factor) derived from helper T cells (type 1). On the other hand, it has an activity to stimulate growth and differentiation of activated B cells. Also, it is referred to as participating in suppression of inflammatory responses in many aspects.

[IL-10 Receptor]

IL-10 receptors on the cell surface mediate activities of IL-10. The IL-10 receptor is a member of an interferon receptor-like subgroup of cytokine receptor family. cDNAs encoding human and mouse interleukin-10 receptors have already been cloned (Nonpatent Document 5: J Immunology Vol. 152, p. 1821-1829; Nonpatent Document 6: PRONAS Vol. 90, p. 11267-11271; Nonpatent Document 7: The EMBO Journal Vol. 16, p. 5894-5903). The IL-10 receptor includes two kinds of polypeptides: IL-10R1 having high affinity with IL-10, and IL-10R2 having low affinity with IL-10.

Furthermore, only the extracellular region of IL-10R1 has been expressed and prepared (Nonpatent Document 8: J. Biol. Chem. Vol. 270, P. 12906-12911). Patent Document 1: U.S. Pat. No. 6,180,370
Nonpatent Document 1: Nature. (1984) Vol. 312, p. 643-6.
Nonpatent Document 2: Nature (1986) Vol. 321, p. 783-792
Nonpatent Document 3: Nature (1988) Vol. 332, 323
Nonpatent Document 4: PRONAS Vol. 88, p. 10535-19539
Nonpatent Document 5: J Immunology Vol. 152, p. 1821-1829,
Nonpatent Document 6: PRONAS Vol. 90, p. 11267-11271
Nonpatent Document 7: The EMBO Journal Vol. 16, p. 5894-5903
Nonpatent Document 8: J. Biol. Chem. Vol. 270, P. 12906-12911

### DISCLOSURE OF THE INVENTION

(1) Conventional antibody drugs necessitate preparation of large quantity of a fusion protein beforehand for administration of the fusion protein to a human body, but expression of the fusion protein still requires a considerable cost.

For the present invention, problems to be solved involve accomplishing a therapy at a low cost by producing a vector that expresses a fusion protein such as an antibody drug, and using the same in gene therapy.

(2) Also, another problem to be solved by the invention is development of an effective antagonist against IL-10, taking into account of the immunosuppressive aspect of IL-10, which hampers particularly the treatment of tumors and the like.

The present inventors developed a vector for recombinant gene therapies which can produce antibody drug in the body by incorporating a gene encoding the antibody drug into a vector for the gene therapy so that the antibody drug can be efficiently administered.

More specifically, the antibody drug (immunoadhesin) in which the extracellular region of the IL-10 receptor 1 was fused with the Fc region of IgG1 was developed.

The invention reduces the cost of conventional antibody drug therapies by providing an expression vector including a DNA that encodes the antibody drug, for the therapies in which the antibody drug is used.

Still further, in another aspect of the invention, a therapeutic drug for a disease mediated by IL-10 is provided by providing a fusion protein in which IL-10 receptor 1 is bound to IgG1, specifically a constant region of IgG1.

The present specification includes the contents described in specification and/or drawings of Japanese Patent Application No. 2003-310601 on which priority of the present application is claimed.

### BRIEF DESCRIPTION OF THE DRAWINGS

FIG. 1 shows a primer list I used in the present invention.

FIG. 2 shows a sequence of IL-10R1/IgG1_{—}1-A.

FIG. 3 shows a sequence of IL-10R1/IgG1_{—}2-A.

FIG. 4 shows a view illustrating a predicted three-dimensional structure of IL-10R1/IgG1_{—}1-A.

FIG. 5 shows a view illustrating a predicted three-dimensional structure of IL-10R1/IgG1_{—}2-A.

FIG. 6 shows a primer list II.

FIG. 7 shows an illustration of the sequence of pVAX1-IL10R1 (EC*).

FIG. 8 shows an illustration of the sequence of pVAX1-IL10R1 (EC)/IgG1-Fc (V51: without hinge).

FIG. 9 shows an illustration of the sequence of pVAX1-IL10R1 (EC)/IgG1-Fc (V52: mutated form hinge **).

FIG. 10 shows an illustration of the sequence of pVAX1-IL10R1 (EC)/IgG1-Fc (V55: wild type hinge ***).

FIG. 11 shows the inhibitory activity of each IL-10 activity, wherein #0 represents pVAX1; #1 represents pVAX1-IL10R1 (V12: EC*); #2 represents pVAX1-IL10R1 (EC)/IgG1 (V51: without hinge); #3 represents pVAX1-IL10R1 (EC)/IgG1 (V15: SSC type hinge); and #4 represents pVAX1-IL10R1 (EC)/IgG1 (V54: CSC type hinge).

FIG. 12 shows an illustration of the sequence of pVAX1-IL10R1 (EC)/IgG1-Fc (V54: CSC type mutated form hinge **).

FIG. 13 shows a view illustrating a predicted three-dimensional structure of IL-10R1_V12.

FIG. 14 shows a view illustrating a predicted three-dimensional structure of IgG1 (Hinge+CH2+CH3)_WT.

FIG. 15 shows a view illustrating a predicted three-dimensional structure of IL10R1-IgG1 (V51: without hinge).

FIG. 16 shows a view illustrating a predicted three-dimensional structure of IL10R1-IgG1 (V52: SSS type mutated form hinge).

FIG. 17 shows a view illustrating a predicted three-dimensional structure of IL10R1-IgG1 (V54: CSC type mutated form hinge).

FIG. 18 shows a view illustrating a predicted three-dimensional structure of IL10R1-IgG1 (V55: CCC type wild type hinge).

### BEST MODE FOR CARRYING OUT THE INVENTION

The present invention provides an antibody drug or an antibody drug candidate, a gene encoding the antibody drug or the antibody drug candidate, and an expression vector of the antibody drug or the antibody drug candidate incorporating the gene encoding the antibody drug or the antibody drug candidate. The present recombinant expression vector can be used for the production of antibody drugs, and gene therapies.

[Fusion Protein: Antibody Drug]

Examples of the antibody drug or the antibody drug candidate for use in the invention include humanized antibodies, and further, fusion proteins (immunoadhesin) in which a constant region of a human antibody is bound to a ligand binding site of a cell surface receptor.

In the invention, the immunoadhesin may involve fusion proteins in which the constant region of a human antibody is fused with a protein other than antibodies, for example, a molecule having a binding action with other molecule such as a receptor, an adhesion factor, or a ligand. More specifically, the immunoadhesin may include fusion antibody proteins in which a constant region of a human antibody is fused with an extracellular region of a cell membrane receptor, suitably, a ligand binding region; or more suitably, fusion proteins to which an IL-10 receptor or an extracellular region thereof, or an extracellular region of IL-10 receptor 1 is fused.

As the constant region of the human antibody which may constitute the immunoadhesin, constant regions of IgG, IgM, and IgA can be utilized. Suitably, the constant regions of IgG can be used. As the constant region of IgG, (A) Fc part, (B) a region including CH2 and CH3, (C) a region including a hinge part, CH2 and CH3, a region where CH1 to CH3 are connected, or the like, and apart or a region which is generated by deletion, addition, substitution, or insertion of one to several amino acids in any of these above regions and which also function as a constant region of an antibody can be used. Specifically, the constant region of IgG1, more specifically, the constant region of IgG1 which can be cloned, for example, from total RNA of B cells, or from SRα-neo1-CD80/CD86/IgFc can be used.

Examples of the protein other than antibodies that may constitute the immunoadhesin include molecules having a binding action (binding ability) with other molecules such as a receptor, an adhesion factor, or a ligand; or fragments of a receptor, an adhesion factor, or a ligand maintaining a binding ability with other molecules; and soluble fragments of the same. Cell membrane receptors, or fragments of their extracellular regions are suitable. Although a variety of membrane protein receptors can be used as the cell membrane receptor, suitably, the IL-10 receptor and an extracellular region thereof, and more suitably, an extracellular region of the IL-10 receptor 1 can be used.

As the extracellular region of the IL-10 receptor 1, suitably, the fragment of amino acids at positions 1 to 235 or amino acids at positions 1 to 228 of the sequence set out in SEQ ID NO: 13 can be selected.

Further examples of the IL-10 receptor 1 extracellular region include arbitrary fragments which include the extracellular region which have a binding ability with IL-10; and also polypeptides in which there has been mutation such as deletion, substitution, addition or insertion of 1 to several amino acids (suitably 1 to 50, further suitably 1 to 20, and more suitably 1 to 10 or 1 to 5 amino acids) in the amino acid position 1-235 in SEQ ID NO: 13 (polypeptide encoded by a fragment of the positions 62 to 766 in SEQ ID NO: 14), and which have a binding activity with IL-10; and polypeptides in which there has been mutation such as deletion, substitution, addition or insertion of 1 to several amino acids (suitably 1 to 50, further suitably 1 to 20, and more suitably 1 to 10 or 1 to 5 amino acids) in the amino acid positions 1 to 228 in SEQ ID NO: 13 (polypeptide encoded by a fragment of the positions 62 to 745 in SEQ ID NO: 14), and which have a binding activity with IL-10.

Examples of the constant region part of antibodies include, when preparation of a fusion protein with the IL-10 receptor 1 is intended, specifically, the Fc part, the region including CH2 (Constant region Heavy chain domain 2) and CH3 (Constant region Heavy chain domain 3), or the region including the hinge part, and CH2 and CH3 of IgG1; and regions in which 1 to several amino acids have been deleted, added, substituted, or inserted, and which function as a constant region of the antibody; preferably, those constituted so that the constant region part of the antibody does not form a dimer; particularly preferably Fc regions of IgG1 having a mutated form hinge yielded by deletion of the hinge part in the Fc part of IgG1, or by mutation of another amino acid (suitably serine) executed so that cysteine in the hinge part does not form a dimer; and specifically, (A) the constant region of IgG1 having a mutated form hinge yielded by mutation of at least two among three cysteine residues in the hinge part into serine residue, and (B) the region including CH2 (Constant region Heavy chain domain 2) and CH3 (Constant region Heavy Chain domain 3).

Preferable examples of the immunoadhesin include fusion proteins in which the extracellular region of the IL-10 receptor 1 is fused with the constant region (CH2, CH3 and the hinge part, or CH2 and CH3) of IgG1 (heavy chain), and particularly preferable are fusion proteins of the extracellular region of the IL-10 receptor 1 with the constant region (CH2 and CH3, or CH2, CH3 and mutated hinge part (the hinge part modified so that the fusion protein does not form a dimer) of IgG1 (heavy chain). Such immunoadhesin can be used as an IL-10 inhibitor that traps the IL-10, and in addition thereto, can be used in regulating the IL-10 activity.

Moreover, the fusion protein of the presently claimed invention may include the fusion proteins having the IL-10 inhibitory activity, constituted from the following contents (1) and (2).

(1) An extracellular region polypeptide of the IL-10 receptor 1 represented by the following (a) or (b):

(a) a polypeptide including amino acids from position 1 to 235 or amino acids from position 1 to 228 in the amino acid sequence of the IL-10 receptor 1 set out in SEQ ID NO: 13;

(b) a polypeptide in which there has been deletion, substitution and/or addition of 1 to several amino acids (suitably, 1 to 50, more suitably 1 to 20, and still more suitably 1 to 10 or 1 to 5 amino acids) in the peptide represented by the amino acid sequence 1 to 235 set out in SEQ ID NO: 13, and which has IL-10 receptor activity, and

(2) An Fc region of IgG1 represented by the following (c), (d) or (e):

(c) a polypeptide encoded by the gene sequence from the base position 70 to 115 to the base position 768 in the IgG1 gene sequence set out in SEQ ID NO: 12;

(d) a polypeptide encoded by the gene sequence from the base position 70 to 115 to the base position 768 in the IgG1 gene sequence set out in SEQ ID NO: 12 in which there has been deletion, substitution, or/and addition of 1 to several amino acids (suitably 1 to 50, further suitably 1 to 20, and more suitably 1 to 10 or 1 to 5 amino acids), and which has activity as an IgG1-Fc fragment;

(e) a polypeptide encoded by the gene sequence from the base position 70 to 115 to the base position 768 in the IgG1 gene sequence set out in SEQ ID NO: 12 in which there has been deletion, substitution, or/and addition of 1 to several amino acids (suitably 1 to 50, further suitably 1 to 20, and more suitably 1 to 10 or 1 to 5 amino acids), and which is a soluble mutated IgG1-Fc fragment that does not form a dimer.

More specifically, the fusion protein of the presently claimed invention may include the following fusion proteins:

(a) a polypeptide including amino acids from position 1 to 235 or amino acids from position 1 to 228 in the amino acid sequence of the IL-10 receptor 1 set out in SEQ ID NO: 13, and (b) the polypeptide encoded by the gene of the bases from position 115 to 768 or bases from position 82 to 768 in the IgG1 gene sequence set out in SEQ ID NO: 12, in which at least two among the G's at the positions 83, 101 and 110 are substituted with C.

[Utility of Fusion Protein (Antibody Drug): Target Disease]

Furthermore, the fusion protein in which the IL-10 receptor 1 is bound to the constant region of IgG1 of the invention can be used as a therapeutic drug or a therapeutic drug candidate for diseases mediated by IL-10. Specifically, for example, antibody drugs (fusion proteins) of the invention in which (1) IL-10 receptor 1 extracellular region is fused with (2-1) a constant region of IgG1 with the hinge part deleted or (2-2) an IgG1 constant region having a mutated hinge part generated by mutation of cysteine in the hinge part with another amino acid so as not to form a dimer can be used for promotion of the activation of killer T cells, and further, for therapies for various cancers including melanoma.

[Antibody Drug Gene]

Examples of the gene encoding the antibody drug which can be incorporated into the vector according to the invention include the aforementioned [Antibody Drug], specifically, nucleic acids such as DNAs or nucleotides encoding the fusion protein (immunoadhesin) in which a humanized antibody, still more specifically the constant region of the human antibody, is bound to a ligand binding site of the cell surface receptor.

Specific examples are genes encoding fusion antibody proteins in which the constant region of the human antibody is fused with the extracellular region, preferably the ligand binding region, of the cell membrane receptor. Moreover, as the cell membrane receptor, a variety of membrane protein receptors can be used, but preferably the IL-10 receptor can be used.

The gene of the presently claimed invention may also include, for example, the following genes (1)-(4).

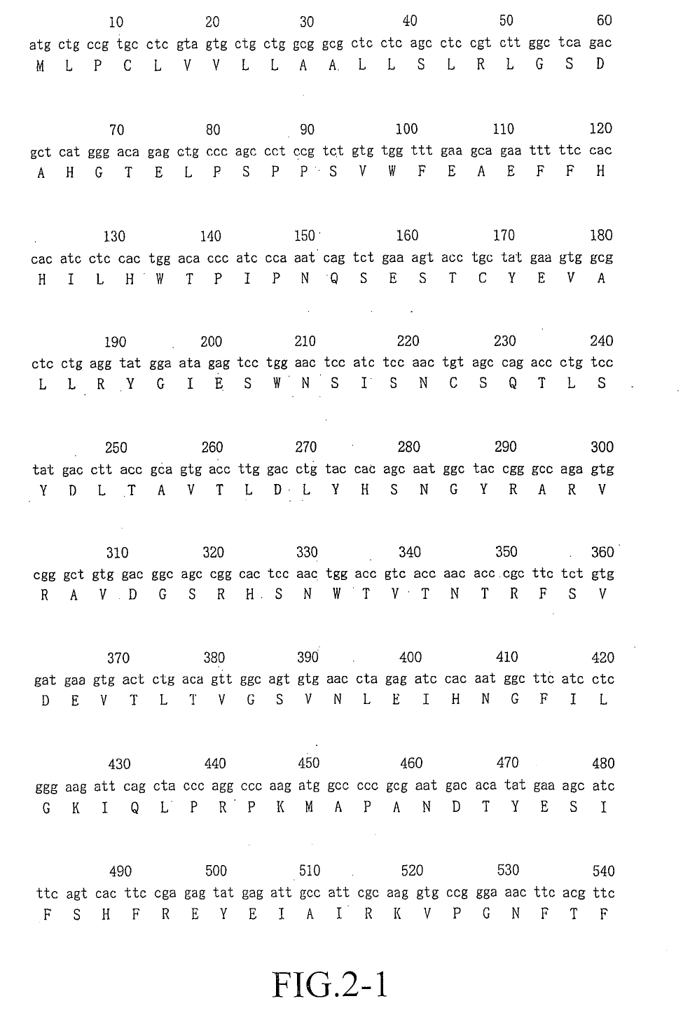

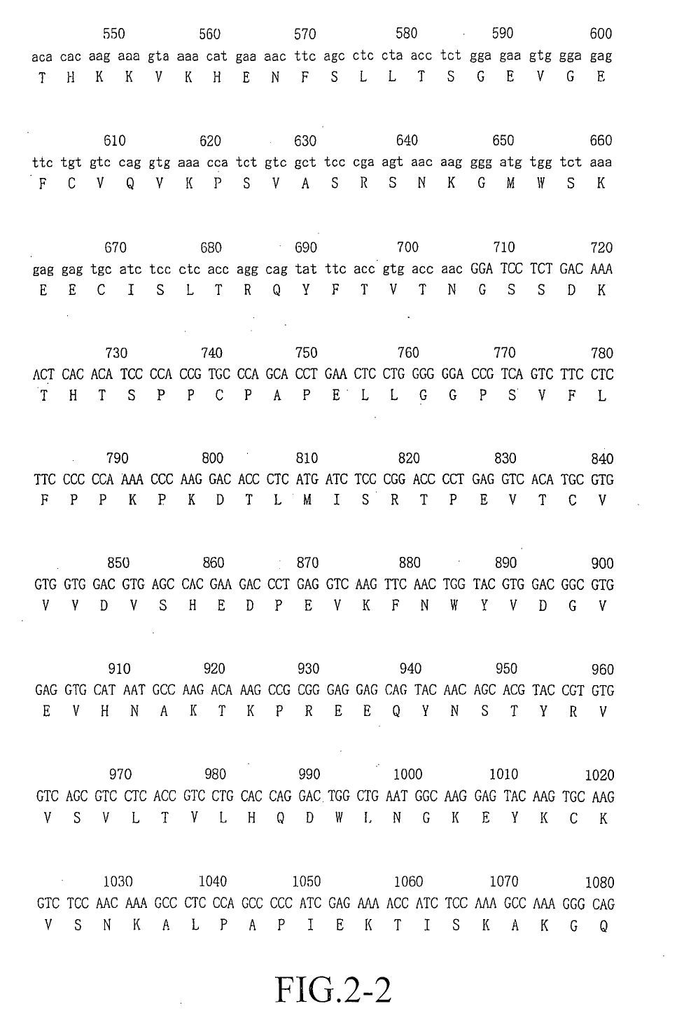

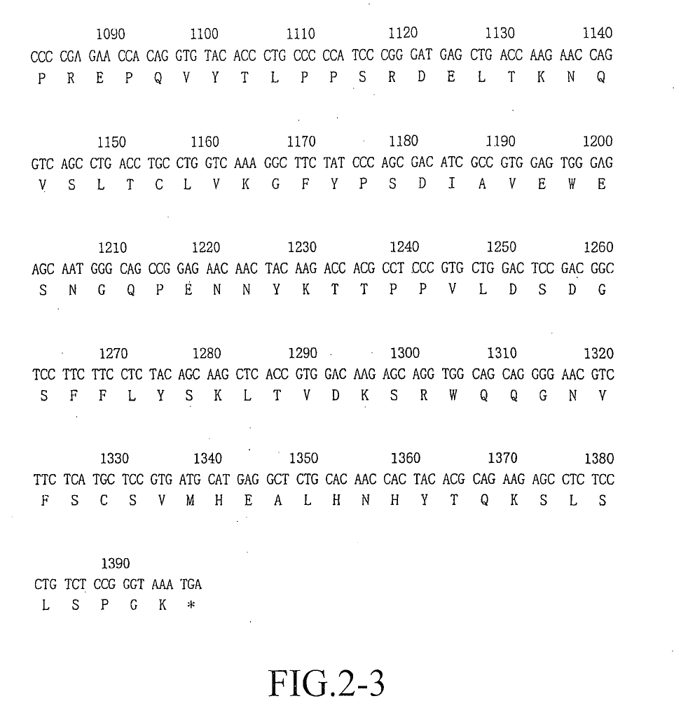

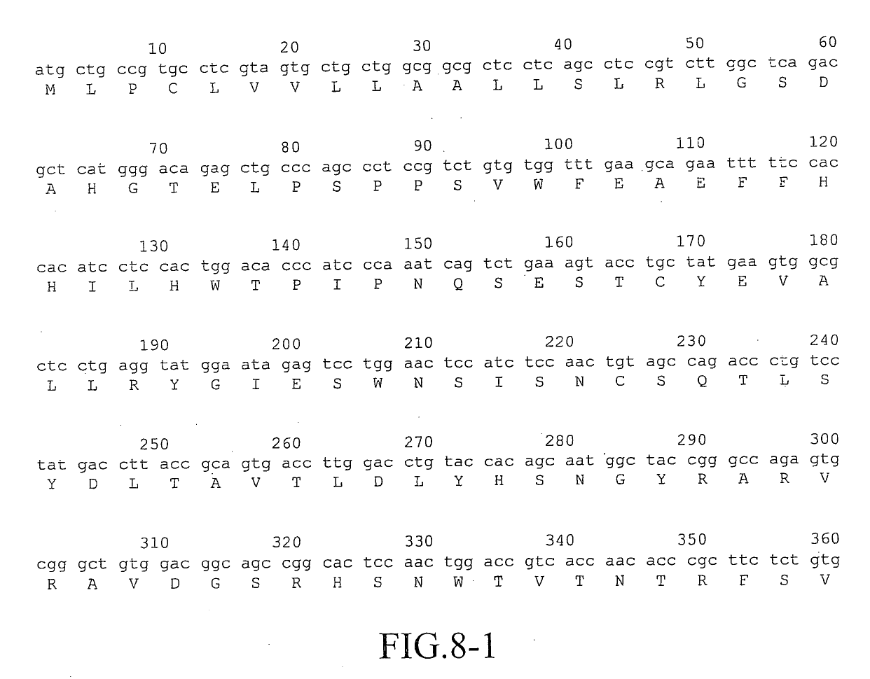

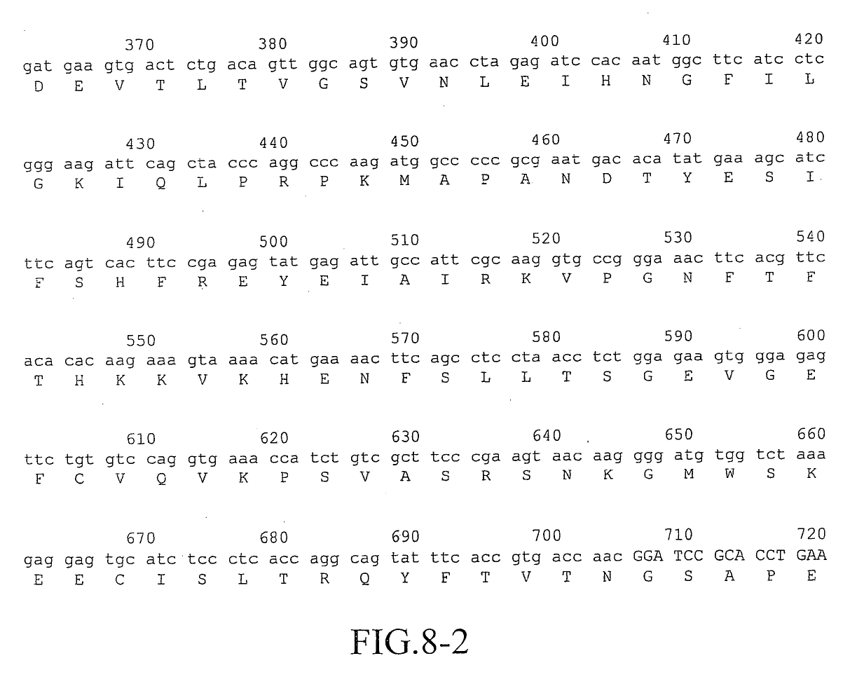

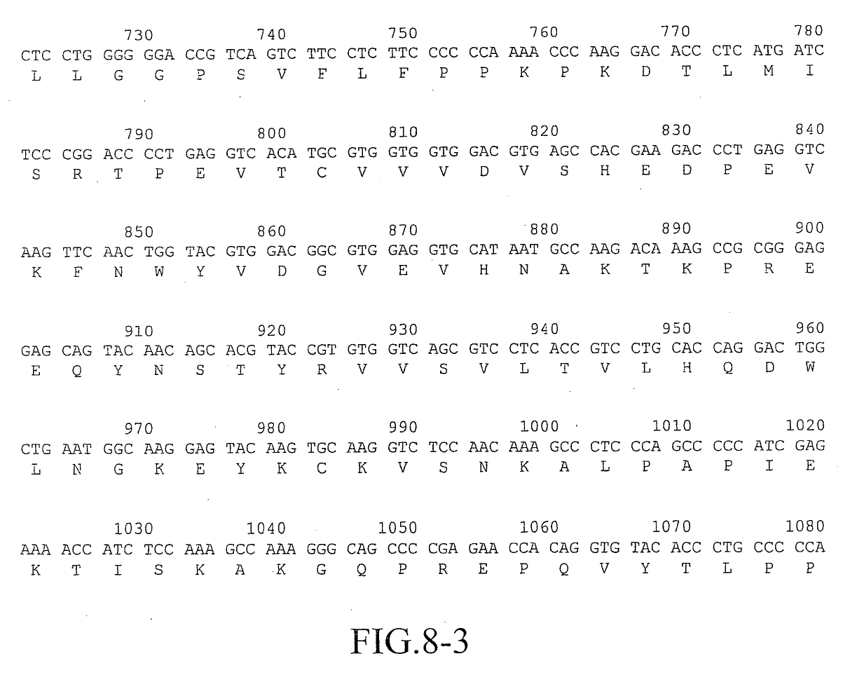

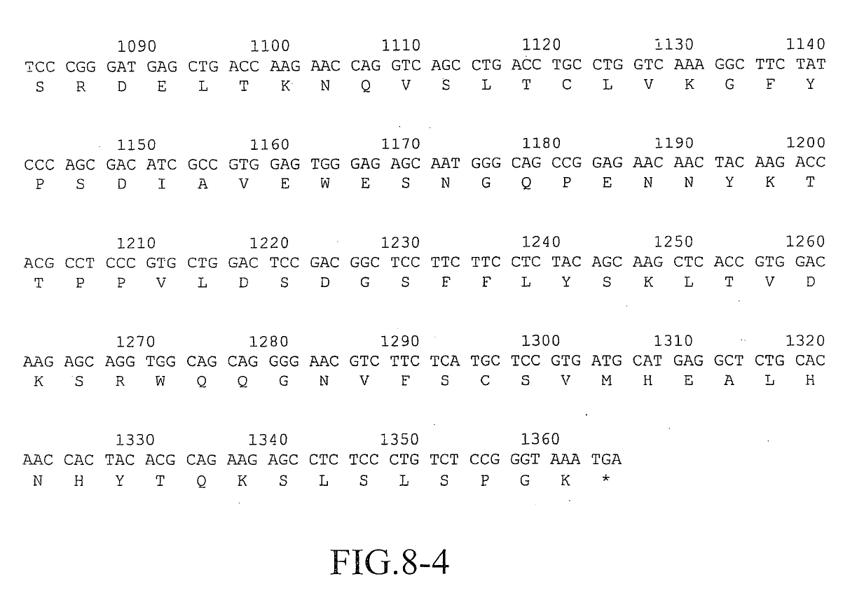

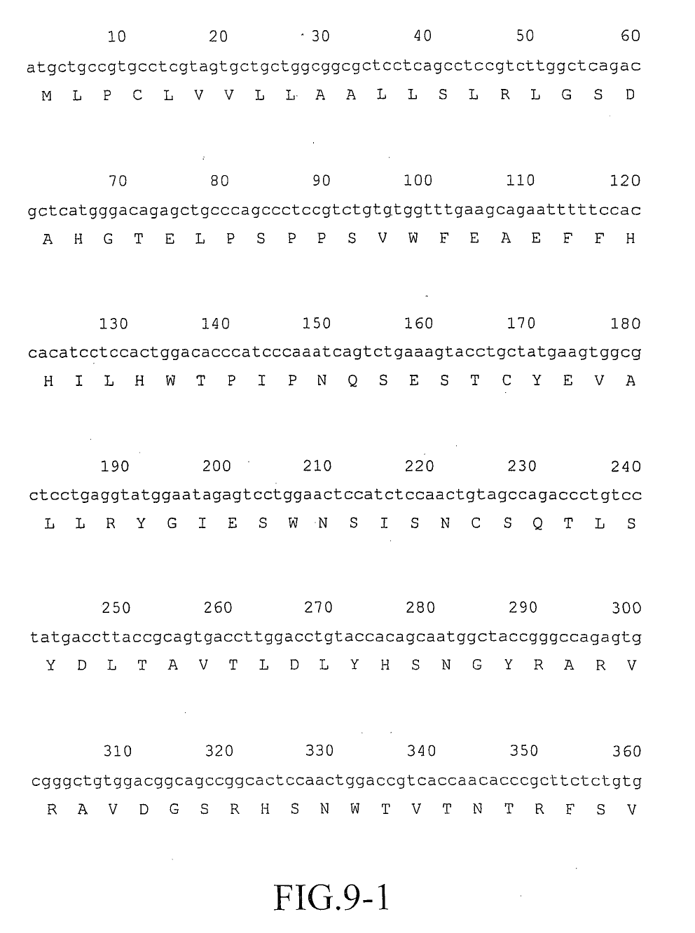

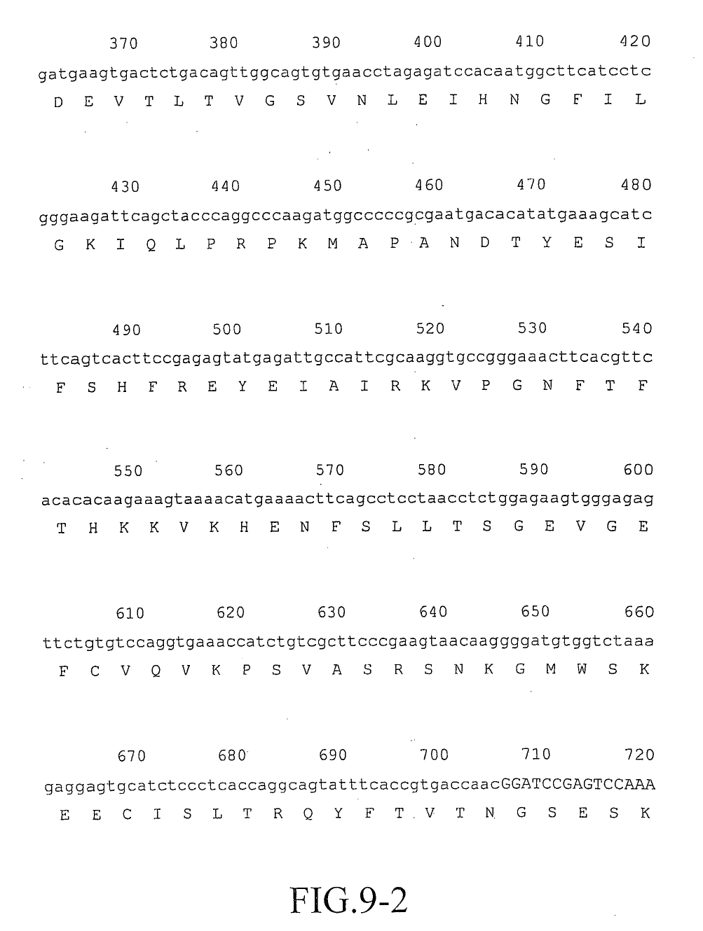

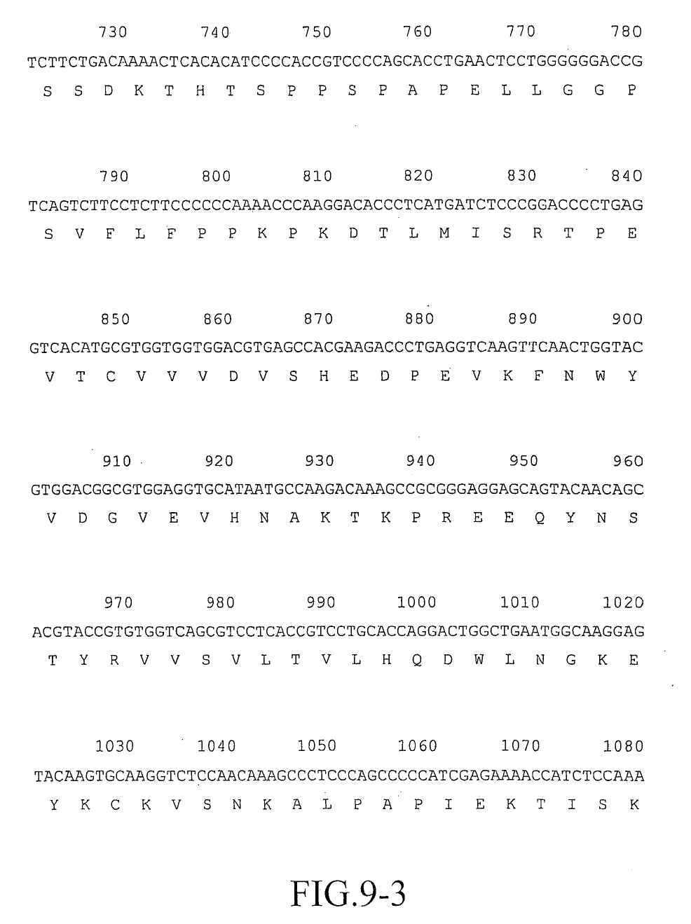

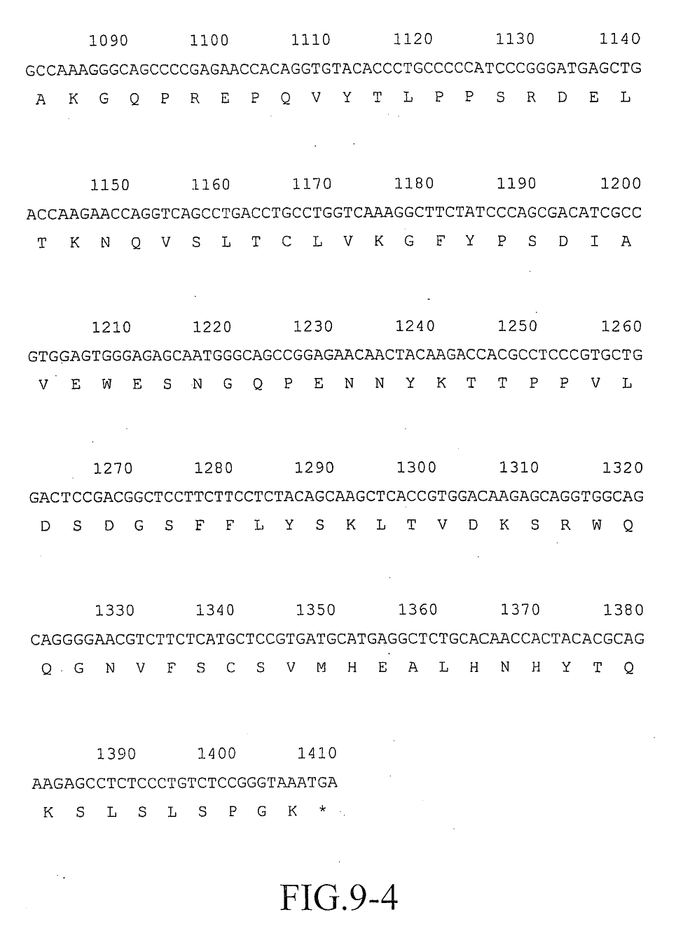

(1) Genes shown in any one of FIG. 2 or 8 to 10 (SEQ ID NO: 7, 17, 19 or 21), preferably FIGS. 8 to 9 (SEQ ID NO: 7, 17 or 19).

(2) Genes encoding polypeptides in which there has been deletion, substitution, and/or addition of 1 to 100, suitably 1 to 20, more suitably 1 to 10 amino acids in the polypeptide shown in any one of FIG. 2 or 8 to 10 (SEQ ID NO: 8, 18, 20 or 22), and which have an IL-10 inhibitory activity.

(3) Genes that hybridize with the gene shown in any one of FIG. 2 or 8 to 10 (SEQ ID NO: 7, 17, 19 or 21), preferably in FIG. 8 or 9 (SEQ ID NO: 7, 17 or 19) under stringent conditions, and which encode polypeptides that have IL-10 inhibitory activity. The stringent conditions imposed may be usual stringent conditions, for example, (A) washing is conducted with a low ion strength, at a high temperature, e.g., conducted with 0.015 M NaCl, 0.0015 M sodium citrate, 0.1% SDS at 50° C., (B) washing is conducted with 50% formaldehyde, 5×SSC (0.75 M NaCl, 0.075 M citric acid), 5.×Denhardt's solution, salmon sperm DNA (50 g/ml), 0.1% SDS, and 10% dextran sulfate at 42° C., further with 0.2×SSC, 0.1% SDS at 42° C., and the like.

(4) Genes that have 60% identity, suitably 80% identity, more suitably 90% identity, particularly suitably 95% identity with the gene shown in any one of FIG. 2 or 8 to 10 (SEQ ID NO: SEQ ID NO: 7, 17, 19 or 21), preferably in FIG. 2 or 8 or 9 (SEQ ID NO: 7, 17 or 19), that do not form a dimer, and which encode polypeptides having IL-10 inhibitory activity.

[Vector]

Examples of the vector (expression vector) which may be used in the invention to administer to humans include a variety of vectors used in gene therapy, e.g., vectors prepared based on adenovirus, adeno associated virus, herpes simplex virus, Sendai virus, or lentivirus, and for example, vectors deficient in replicating function can be used. Furthermore, a plasmid which replicates and proliferates in prokaryote, and which causes transient expression in mammalian cells can be also used.

Suitably, for enabling gene therapies on humans, pVAX1 available from Invitrogen Corporation which has been already certified by the U.S. Food and Drug Administration can be used as a host vector.

The antibody drug gene of the invention can be prepared in the form of a recombinant vector by recombination into the aforementioned expression vector (host vector). The recombinant vector can be used for expression of the antibody drug and/or for gene therapy.

[Method of Administration]

(1) The prepared antibody drug can be administered by, for example, intravenous injection.

(2) The prepared recombinant antibody drug expression vector for gene therapy can be administered by, for example, introducing it into a lipid vesicle membrane such as liposome or allowing it to coexist with phospholipid, followed by suspending in a common injection buffer and then intramuscular injection, intravenous injection, subcutaneous injection or the like. The amount of the vector administered may be that employed in common gene therapy; for example, when a recombinant adeno vector is administered to humans, it may be administered in an amount of 1×10⁹ to 1×10¹² Pfu (J Clin Oncol. 2002 Mar. 15: 20(6): 1562-9.).

For example, the recombinant antibody drug expression vector incorporated into pVAX1 can be used by intramuscular injection after diluting in an appropriate buffer for injection.

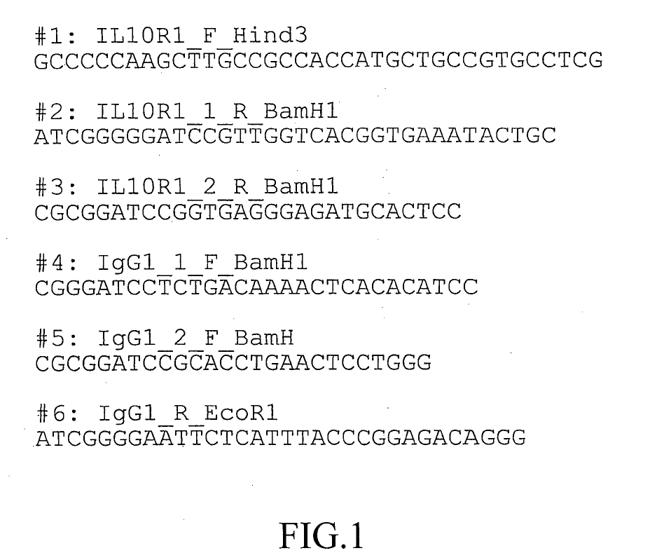

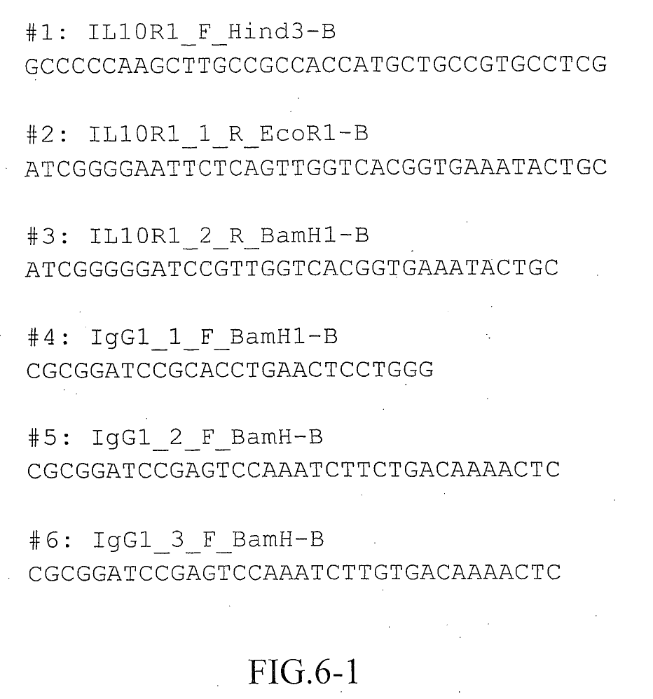

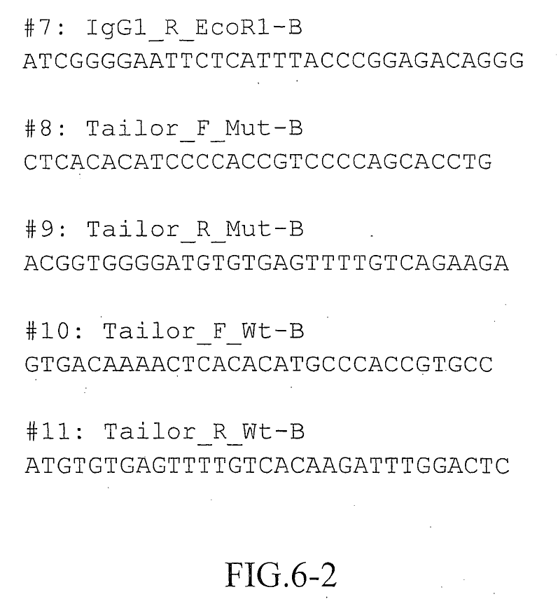

The following Examples are for illustration purposes, and the presently claimed invention is not limited thereto. Herein, Examples 1 to 4 constitute Example Group A, and Examples 5 to 8 constitute Example Group B. The primer according to Example Group A and the primer according to Example Group B mean the primers shown in respective lists for each (Example Group A in FIG. 1; and Example Group B in FIG. 6).

### EXAMPLE 1

### Preparation of cDNA of IL-10R1 Extracellular Region

(1) Preparation of Primer

The primers for excising the IL10R1 extracellular region were designed so that (1) the positions 62 to 766 in SEQ ID NO: 14 of the cDNA encoding the IL-10 receptor can be excised as the extracellular region 1, and (2) the positions 62 to 745 in SEQ ID NO: can be excised as the region 2.

The designed primers shown in FIG. 1 are (1) #1 (SEQ ID NO: 1) as the forward primer and #2 (SEQ ID NO: 2) as the reverse primer for excision of IL-10R1_{—}1-A (region 1), and (2) #1 (SEQ ID NO: 1) as the forward primer and #3 (SEQ ID NO: 3) as the reverse primer for excision of IL-10R1_{—}2-A (region 2).

(2) Preparation of cDNA

Total RNA of Human T-Cell Leukemia (Jurkat) was collected. According to an RT-PCR method, cDNA of the IL-10R1_{—}1-A region was obtained using the primers #1 and #2, and cDNA of the IL-10R1_{—}2-A region was obtained using #1 and #3.

### EXAMPLE 2

### Preparation of IgG1 (Fc) Site

(1) Preparation of Primer

The primers for excising the Fc region of IgG1 were designed so that (A) the positions from 70 to 768 of IgG1 (SEQ ID NO: 12) can be excised in the case of the Fc region 1, and so that (B) the position 115-768 in SEQ ID NO: 12 can be excised in the case of the Fc region 2.

The thus designed primers are shown in FIG. 1: (1) a forward primer, #4 (SEQ ID NO: 4), and a reverse primer, #6 (SEQ ID NO: 6), for excision of IgG-Fc_{—}1-A (region 1), and (2) a forward primer, #5 (SEQ ID NO: 5), and a reverse primer, #6 (SEQ ID NO: 6), for excision of IgG1-Fc2-A (region 2).

(2) Preparation of cDNA

For the IgG1_Fc sites (IgG1-Fc_{—}1-A region and IgG1-Fc_{—}2-A region), cDNA of the IgG1-Fc_{—}1-A region (IgG1_{—}1-A) was obtained using the primers #4 and #6, and cDNA of the IgG1-Fc_{—}2-A region (IgG1_{—}2-A) was obtained using #5 and #6, respectively, from SRα-neo1-CD80/CD86/IgFc (gift from assistant professor David B. Weiner, University of Pennsylvania, School of Medicine, Department of Pathology)

### EXAMPLE 3

### Production of Expression Vector for Gene Therapy of IL-10R1/IgG1-A

A binding site was produced in the host vector pVAX1 (Invitrogen Corporation), using restriction enzymes HindIII and EcoRI. The IL10R1 extracellular regions (IL-10R1_{—}1-A and IL-10R1_{—}2-A) prepared in Example 1 were subjected to end processing with restriction enzymes HindIII and BamHI, and the IgG1_Fc sites (IgG1-Fc_{—}1-A region and IgG1-Fc_{—}2-A region) prepared in Example 2 were subjected to end processing with restriction enzymes BamHI and EcoRI. (1) IL-10R1_{—}1-A and IgG1_{—}1-A were bound to the binding site of pVAX to construct pVAX1-IL10R1/IgG1_{—}1-A (V15: SSCtype hinge), and (2) IL-10R1_{—}2 and IgG1_{—}2 were bound to the binding site of pVAX to construct pVAX1-IL10R1/IgG1_{—}2-A (V50: without hinge). An SSC type hinge is characterized in that two among the three cysteine residues in the hinge part are mutated into serine residues.

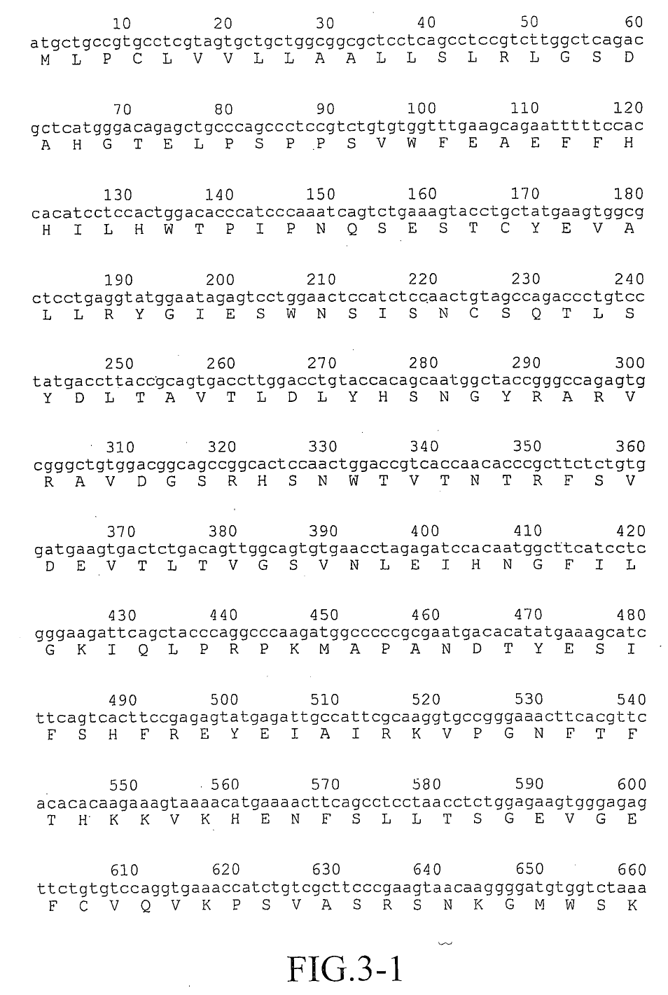

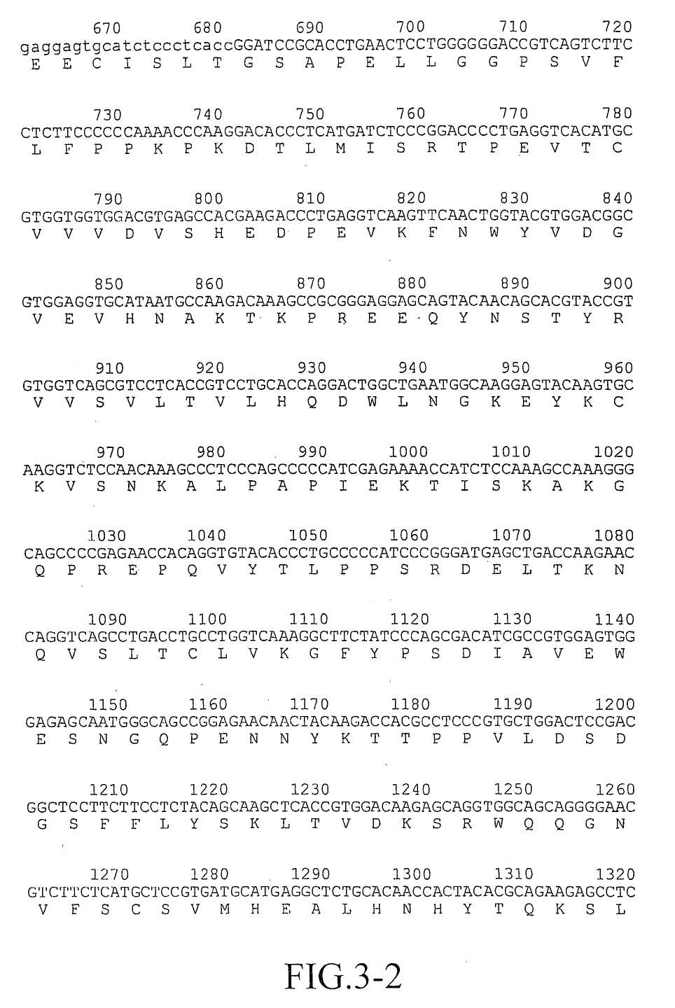

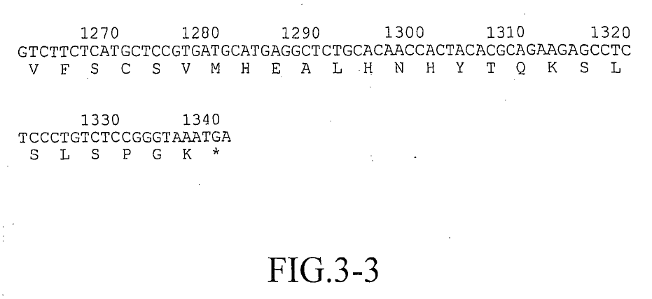

Accordingly, the expression vectors for gene therapy (pVAX1-IL10R1/IgG1_{—}1-A and pVAX1-IL10R1/IgG1_{—}2-A) were constructed. The protein expressed from pVAX1-IL10R1/IgG1_{—}1-A is set out in SEQ ID NO: 8; the gene sequence thereof is shown in SEQ ID NO: 7; and further, both are shown in FIG. 2 in parallel. Also, the protein expressed from pVAX1-IL10R1/IgG1_{—}2-A is shown in SEQ ID NO: 10; the gene sequence thereof is set out in SEQ ID NO: 9; and further, both are shown in FIG. 3 in parallel.

Verification of the base sequence of the constructed expression vector for gene therapy was carried out with a sequence analyzer, and it was found to be 100% correct.

### EXAMPLE 4

### Prediction of Three-Dimensional Structure of IL-10R1/IgG1

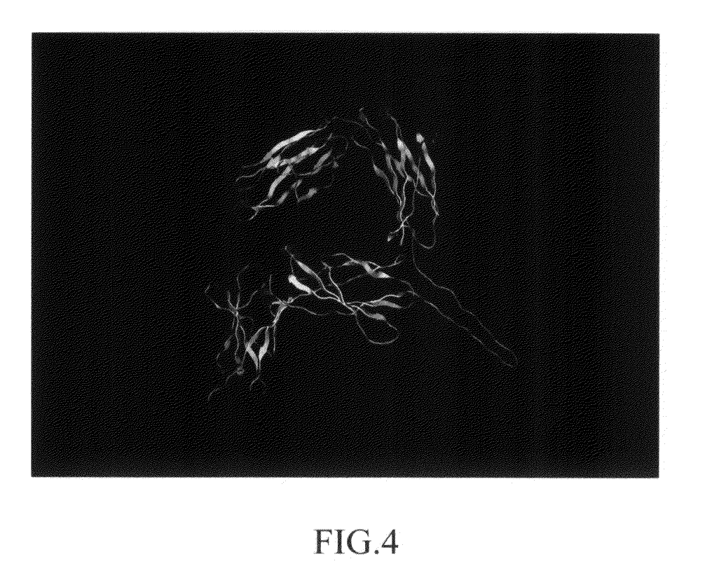

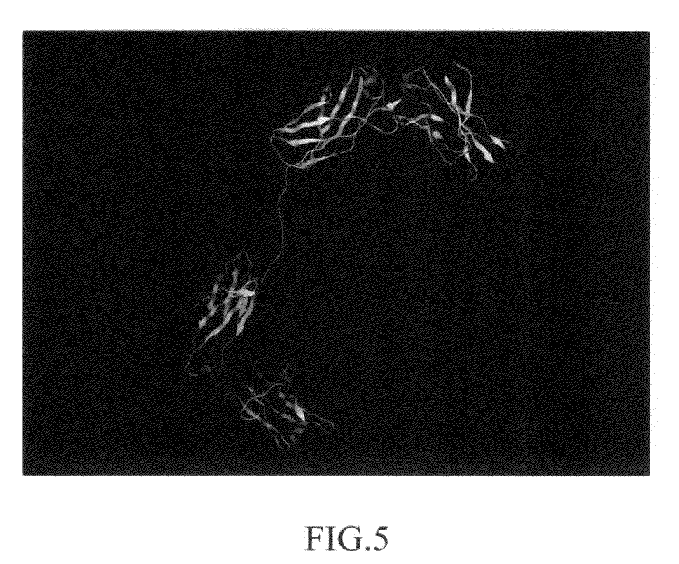

The three-dimensional structures of the proteins (IL10R1/IgG1_{—}1-A and IL10R1/IgG1_{—}2-A) expressed from the two kinds of the expression vectors that were constructed (pVAX1-IL10R1/IgG1_{—}1-A and pVAX1-IL10R1/IgG1_{—}2-A) are shown in FIG. 4 and FIG. 5. These three-dimensional structures were produced with a computation software for chemistry (MOE, Ver. 2003.02, CCG Inc., Montreal).

### EXAMPLE 5

### Preparation of cDNA of IL-10R1 Extracellular Region

(1) Preparation of Primer:

The designed primers are shown in FIG. 6,

(1) the forward primer #1: IL10R1_F_Hind3-B (SEQ ID NO: 1: GCCCCCAAGCTTGCCGCCACCATGCTGCCGTGCCTCG) and the reverse primer #2: IL10R1_{—}1_R_EcoR1-B (SEQ ID NO: 23: ATCGGGGAATTCTCAGTTGGTCACGGTGAAATACTGC) for excision of IL-10R1 (EC*: with a stop codon introduced so as to express only the extracellular region),

(2) the forward primer #1: IL10R1_F_Hind3-B (SEQ ID NO: 1: GCCCCCAAGCTTGCCGCCACCATGCTGCCGTGCCTCG) and the reverse primer #3: IL10R_{—}2_R_BamH1 (SEQ ID NO: 2: ATCGGGGGATCCGTTGGTCACGGTGAAATACTGC) for excision of IL-10R1 (EC: for use in binding with the Fc region of IgG1).

(2) Preparation of cDNA

Total RNA of Human T-Cell Leukemia (Jurkat) was collected. By the RT-PCR method, cDNA of IL-10R1 (EC*) was obtained using the primers #1 and #2 shown in FIG. 6, and cDNA of IL-10R1 (EC) was obtained using #1 and #3.

### EXAMPLE 6

### Preparation of IgG1 (Fc) Site

(1) Preparation of Primer

The primers for excising the Fc region of IgG1 were designed so that (i) the position 115-768 of IgG1 (SEQ ID NO: 12) can be excised, obtaining the Fc region 1 (without hinge part).

(ii) the position 82-768 of IgG1 (SEQ ID NO: 12) can be excised from SRα-neo1-CD80/CD86/IgFc to obtain the Fc region (SSS type mutated form hinge part) when accompanied by mutation of cysteine (codons 82 to 85, 100 to 102, and 109 to 111) to serine.

(iii) the position 82-768 of IgG1 (SEQ ID NO: 12) can be excised from SRα-neo1-CD80/CD86/IgFc to obtain the Fc region (CSC type mutated form hinge part) when accompanied by mutation of cysteine (codon 82-85, and codon 109-111) to serine.

(iv) the position 82-768 of IgG1 (SEQ ID NO: 12) can be excised from SRα-neo1-CD80/CD86/IgFc to obtain the Fc region (wild type hinge part).

Thus designed primers are shown in FIG. 6:

(A) the forward primer #4: IgG1_{—}1_F_BamH1-B (SEQ ID NO: 5: CGCGGATCCGCACCTGAACTCCTGGG) and the reverse primer #7: IgG1_R_EcoR1-B (SEQ ID NO: 6: ATCGGGGAATTCTCATTTACCCGGAGACAGGG) for excision of IgG-Fc_{—}1 (region 1: without hinge part);

(B) the forward primer #5: IgG1_{—}2_F_BamH-B (SEQ ID NO: 24: CGGGATCCTCTGACAAAACTCACACATCC) and the reverse primer #7: IgG1_R_EcoR1-B (SEQ ID NO: 6: ATCGGGGAATTCTCATTTACCCGGAGACAGGG) for excision of IgG1-Fc_{—}2-B (region 2: SSS type mutated form hinge part), and further, the forward primer #8: Tailor_F_Mut-B (SEQ ID NO: 25: CTCACACATCCCCACCGTCCCCAGCACCTG) and the reverse primer #9: Tailor_R_Mut-B (SEQ ID NO: 26: ACGGTGGGGATGTGTGAGTTTTGTCAGAAGA) for modification;

(C) the forward primer #6: IgG1_{—}3_F_BamH-B (SEQ ID NO: 27: CGCGGATCCGAGTCCAAATCTTGTGACAAAACTC) and the reverse primer #7: IgG1_R_EcOR1-B (SEQ ID NO: 6: ATCGGGGAATTCTCATTTACCCGGAGACAGGG) for excision of IgG1-Fc_{—}3 (region 3: CSC type hinge part); and

(D) the forward primer #6: IgG1_{—}3_F_BamH-B (SEQ ID NO: 27: CGCGGATCCGAGTCCAAATCTTGTGACAAAACTC) and the reverse primer #7: IgG1_R_EcOR1-B (SEQ ID NO: 6: ATCGGGGAATTCTCATTTACCCGGAGACAGGG) for excision of IgG1-Fc_{—}3 (region 3: wild type hinge part), and further, the forward primer #10: Tailor_F_Wt-B (SEQ ID NO: 28: GTGACAAAACTCACACATGCCCACCGTGCC) and the reverse primer #11: Tailor_R_Wt-B (SEQ ID NO: 29: ATGTGTGAGTTTTGTCACAAGATTTGGACTC) for modification.

(2) Preparation of cDNA

For the IgG1_Fc sites (IgG1-Fc (without hinge), IgG1-Fc (mutated form hinge) and IgG1-Fc (wild type hinge)), cDNA of IgG1-Fc (without hinge) was obtained from SRα-neo1-CD80/CD86/IgFc using the primers #4 and #7 in FIG. 6; cDNA of IgG1-Fc (SSS type mutated form hinge) was obtained using the primers #5 and #7 in FIG. 6, and additionally the primers #8 and #9 in FIG. 6; cDNA of IgG1-Fc (CSC type hinge) using the primers #6 and #7 in FIG. 6; and cDNA of IgG1-Fc (wild type hinge) was obtained using the primers #6 and #7 in FIG. 6, and additionally the primers #10 and #11 in FIG. 6.

### EXAMPLE 7

### Production of Expression Vector for IL-10R1/IgG1 Gene Therapy

A binding site was produced in the host vector pVAX1 (Invitrogen Corporation), using restriction enzymes HindIII and EcoRI. The IL10R1 extracellular regions (IL-10R1(EC*)1 and IL-10R1 (EC)) prepared in Example 5 were subjected to end processing with restriction enzymes HindIII and BamHI, and the IgG1_Fc sites (IgG1-Fc (without hinge), IgG1-Fc (mutated form hinge) and IgG1-Fc (wild type hinge)) prepared in Example 6 were subjected to end processing with restriction enzymes BamHI and EcoRI.

(A) V12: pVAX1-IL10R1 (EC*) was prepared by incorporating IL10R1(EC*), which had been treated with the aforementioned restriction enzymes, into pVAX1. Primers #1 and #2 in FIG. 6 were used. V52: (B) V51: pVAX1-IL10R1 (EC)/IgG1-Fc (V51: without hinge) was prepared by incorporating IL10R1 (EC) and IgG1-Fc (without hinge), which had been treated with the aforementioned restriction enzymes, into pVAX1.

(C) V52: pVAX1-IL10R1 (EC)/IgG1-Fc (V52: SSS type mutated form hinge **) was prepared by incorporating IL10R1 (EC) and IgG1-Fc (SSS type mutated form hinge **), which had been treated with the aforementioned restriction enzymes, into pVAX1. A SSS type mutated form hinge is characterized in that three cysteine residues in the hinge part are substituted with three serine residues.

(D) V54: pVAX1-IL10R1 (EC)/IgG1-Fc (V54: CSC type mutated form hinge **) was prepared by incorporating IL10R1 (EC) and IgG1-Fc (CSC type mutated form hinge **), which had been treated with the aforementioned restriction enzymes, into pVAX1. A CSC type mutated form hinge is characterized in that among three cysteine residues in the hinge part, the two on the upstream and downstream side are substituted with serine residues.

(E) V55: pVAX1-IL10R1 (EC)/IgG1-Fc (V55: wild type hinge ***) was prepared by incorporating IL10R1 (EC) and IgG1-Fc (wild type hinge ***), which had been treated with the aforementioned restriction enzymes, into pVAX1.

Accordingly, gene expression vectors (pVAX1-IL10R1 (EC*), pVAX1-IL10R1 (EC)/IgG1-Fc (V51: without hinge), pVAX1-IL10R1 (EC)/IgG1-F6 (V52: SSS type mutated form hinge **), V54: pVAX1-IL10R1 (EC)/IgG1-Fc (CSC type mutated form hinge **), and pVAX1-IL10R1 (EC)/IgG1-Fc (V55: wild type hinge ***)) were constructed.

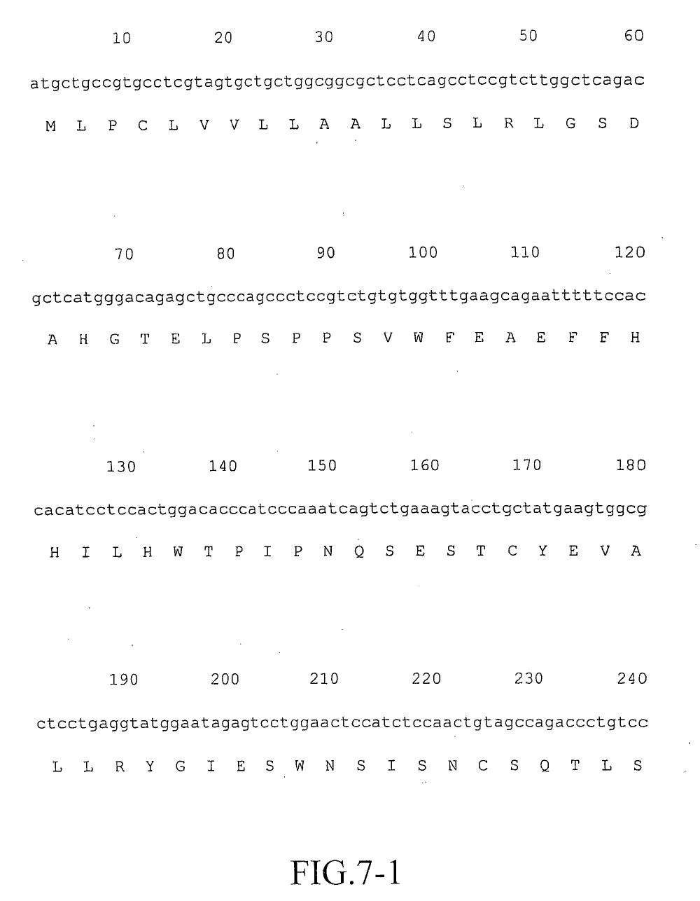

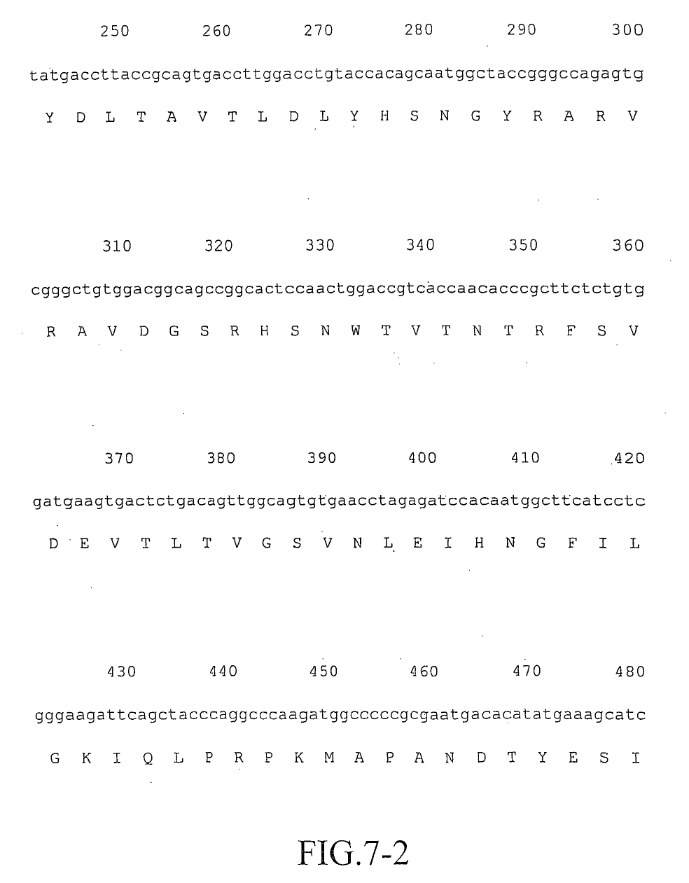

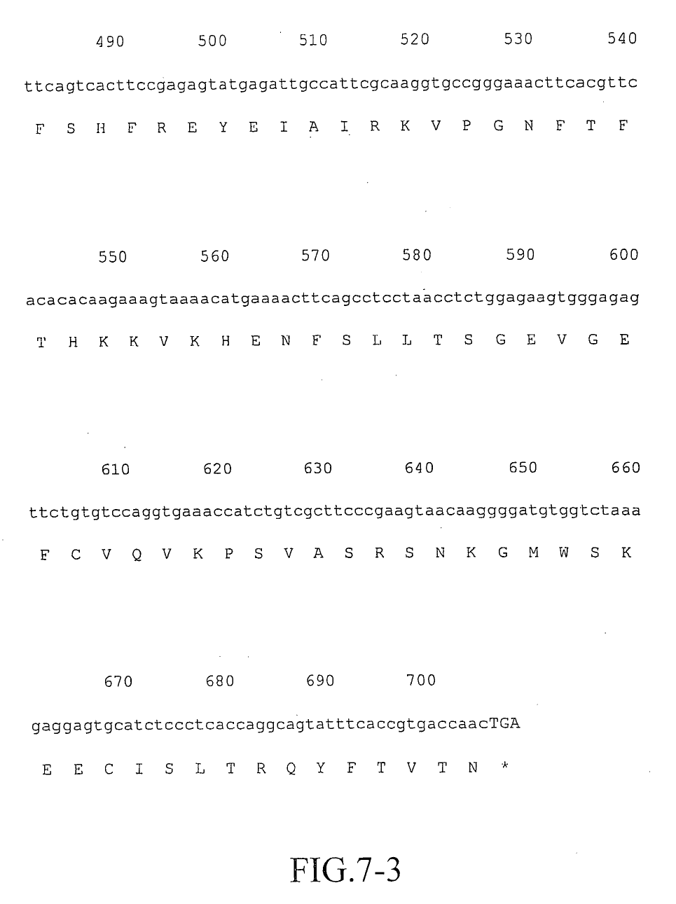

The protein expressed from pVAX1-IL10R1 (EC*) is shown in SEQ ID NO: 15; the gene thereof is shown in SEQ ID NO: 16; and further, both are shown in FIG. 7 (FIG. 7-1 to FIG. 7-3) in parallel.

The protein expressed from pVAX1-IL10R1 (EC)/IgG1-Fc (V51: without hinge) is shown in SEQ ID NO: 18; the gene sequence thereof is set out in SEQ ID NO: 17; and further, both are shown in FIG. 8 (FIG. 8-1 to FIG. 8-4) in parallel.

The protein expressed from pVAX1-IL10R1 (EC)/IgG1-Fc (V52: SSS type mutated form hinge **) is shown in SEQ ID NO: 20; the gene sequence thereof is set out in SEQ ID NO: 19; and further, both are shown in FIG. 9 (FIG. 9-1 to FIG. 9-4) in parallel.

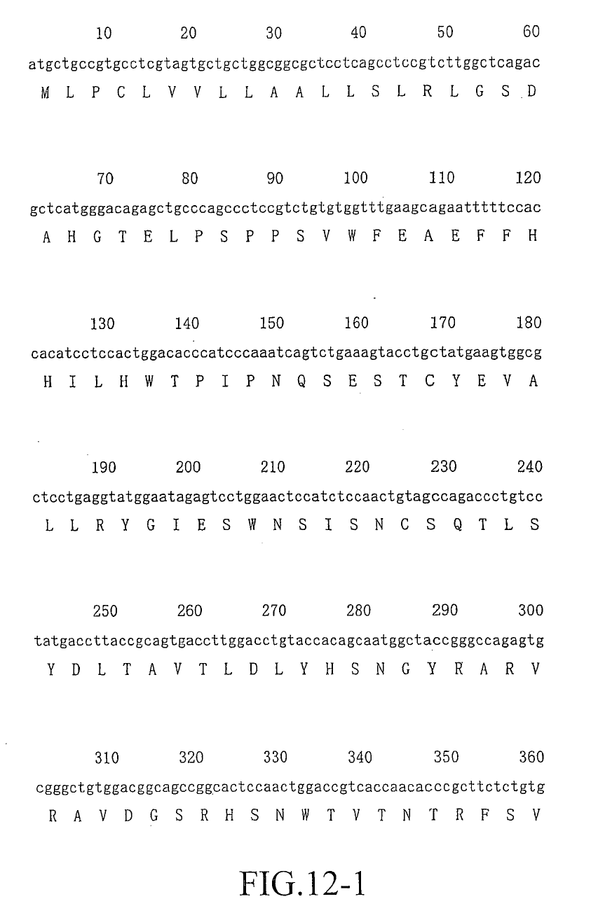

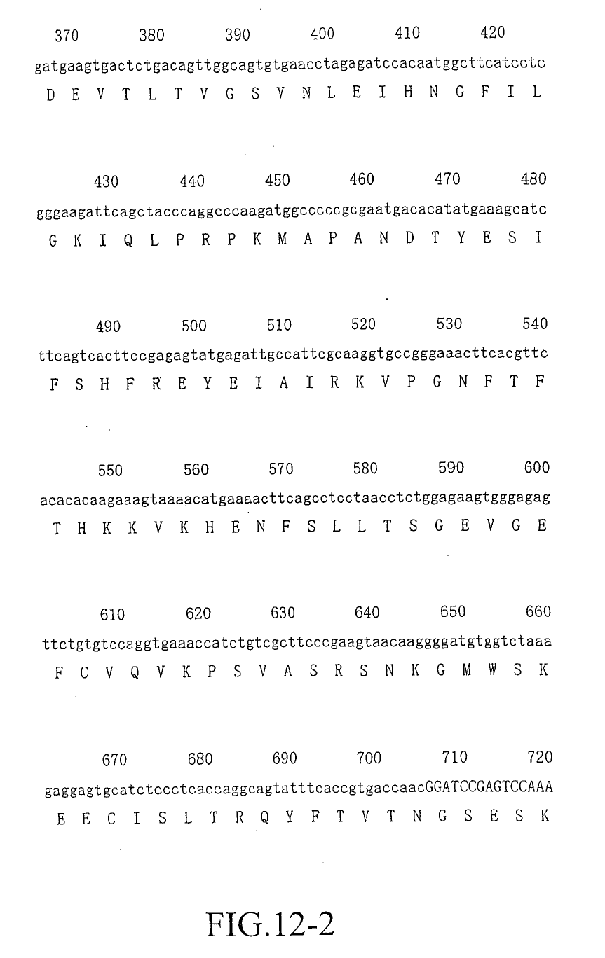

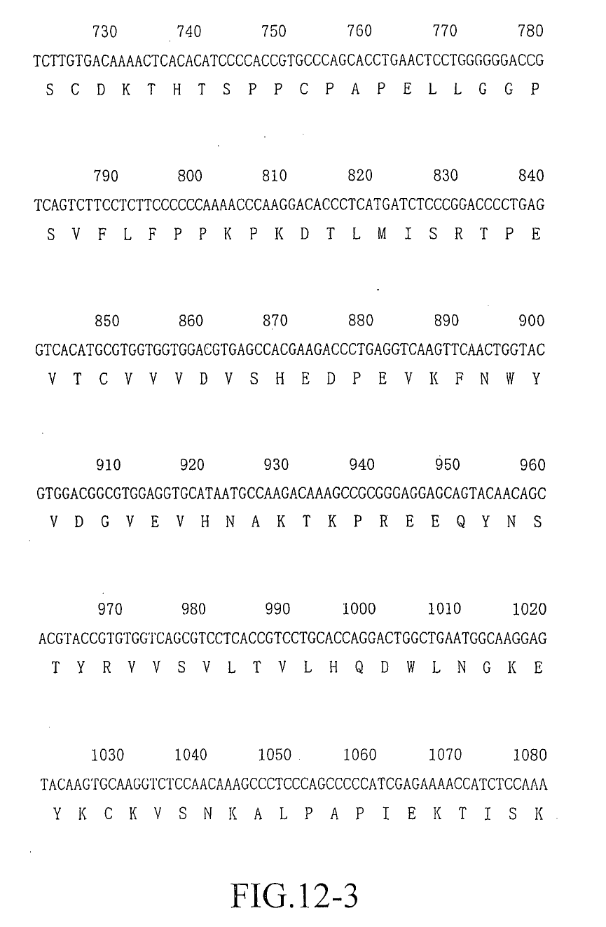

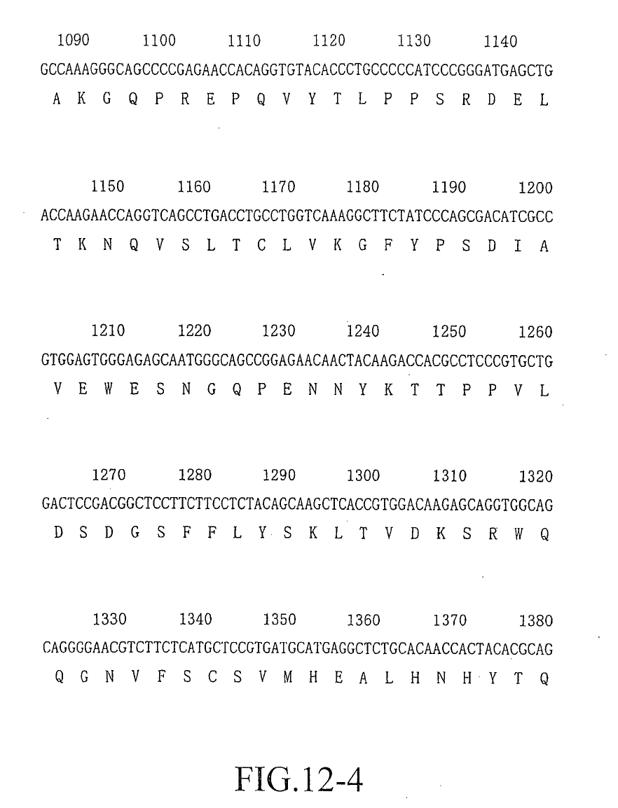

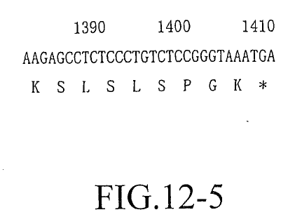

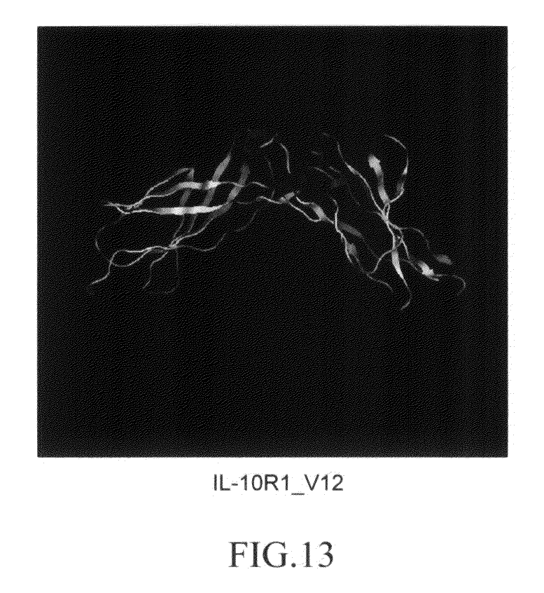

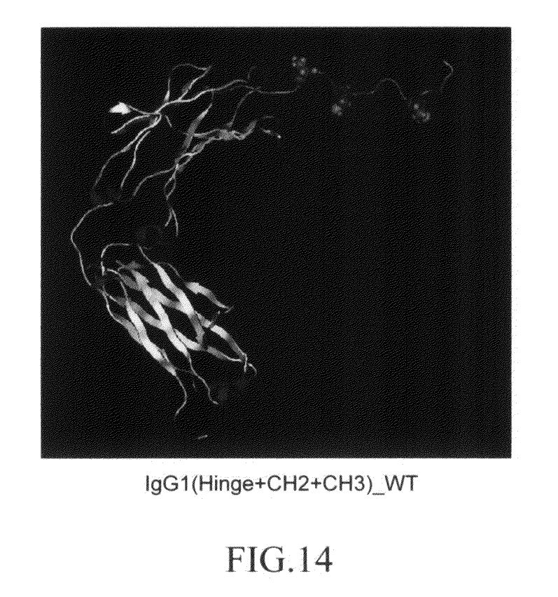

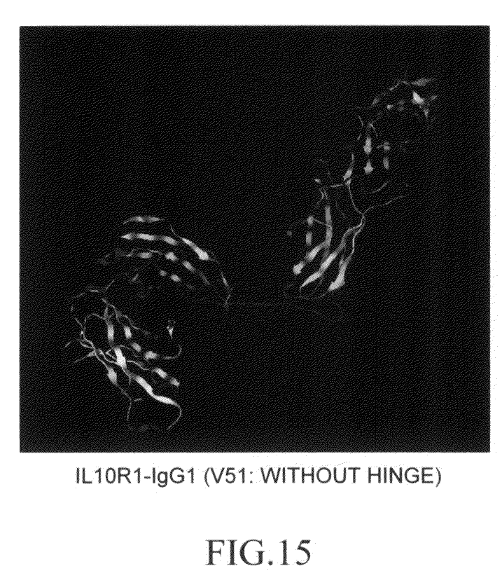

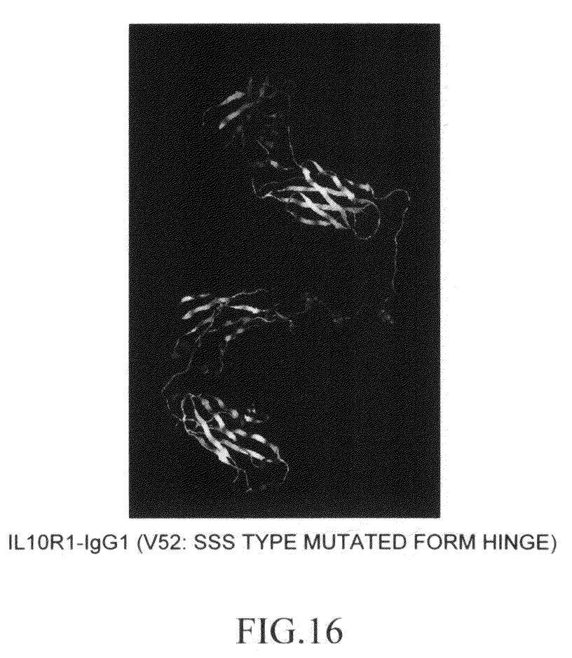

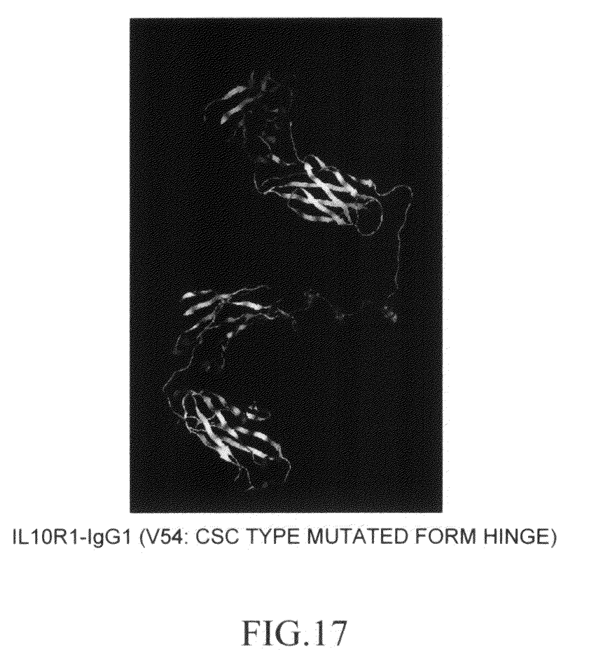

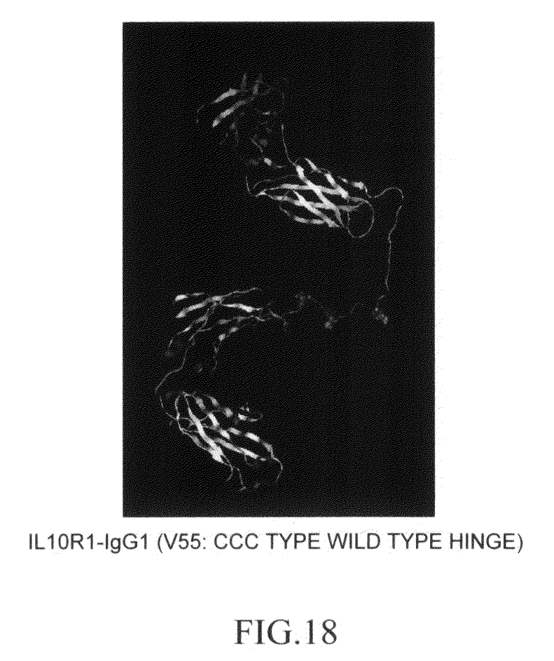

The protein expressed from pVAX1-IL10R1 (EC)/IgG1-Fc (V54: CSC type mutated form hinge **) is shown in SEQ ID NO: 30; the gene sequence thereof is set out in SEQ ID NO: 32; and further, both are shown in FIG. 12 (FIG. 12-1 to FIG. 12-5) in parallel.

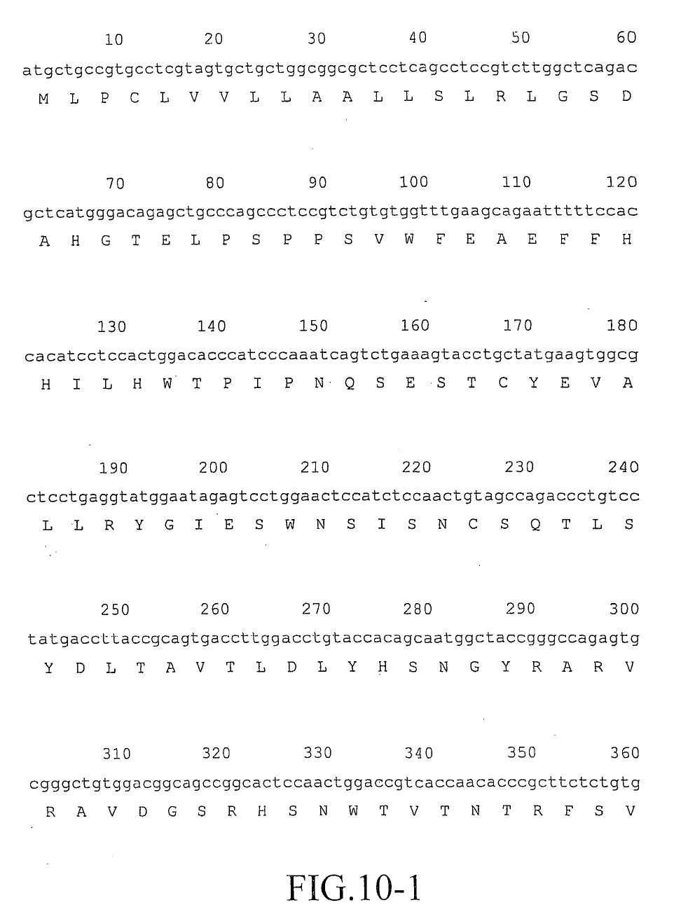

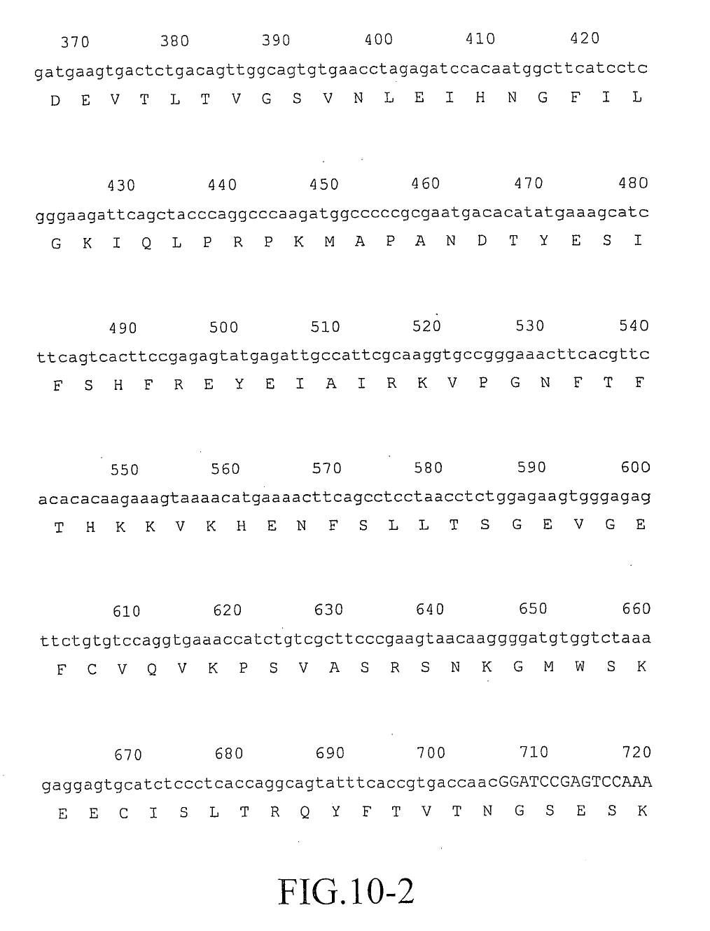

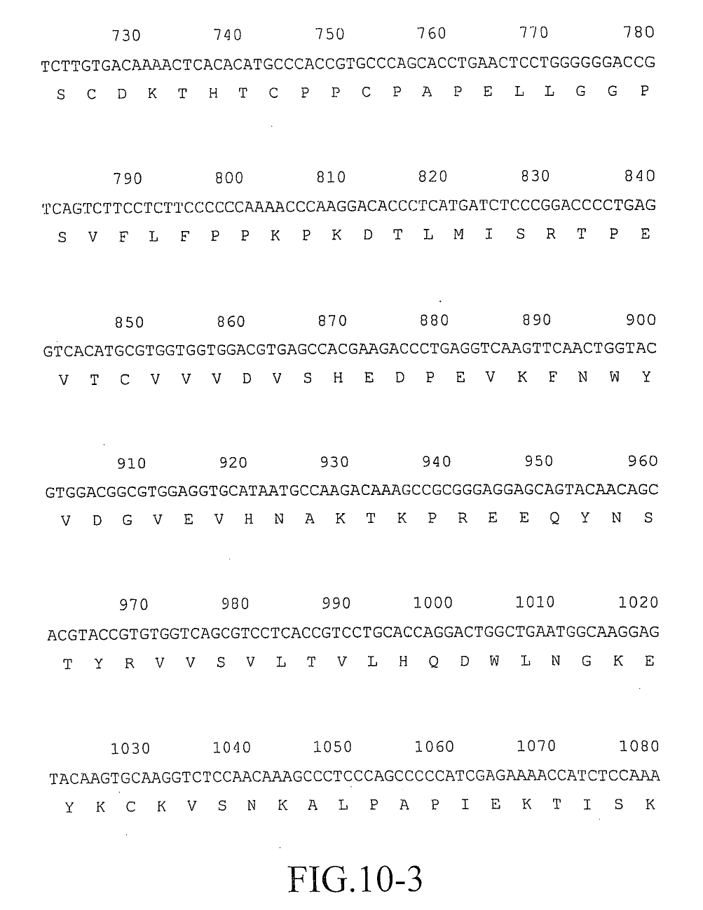

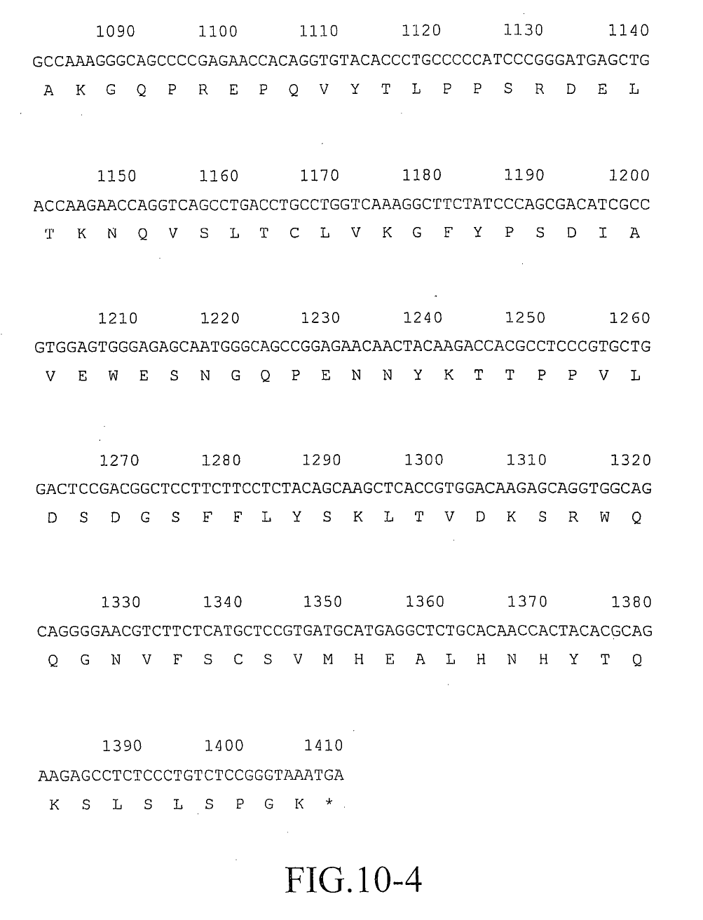

The protein expressed from pVAX1-IL10R1 (EC)/IgG1-Fc (V55: wild type hinge ***) is shown in SEQ ID NO: 22; the gene thereof is shown in SEQ ID NO: 21; and further, both are shown in FIG. 10 (FIG. 10-1 to FIG. 10-4) in parallel.

Verification of the base sequence of the constructed gene expression vector was carried out with a sequence analyzer, and it was found to be 100% correct.

### EXAMPLE 8

(1) Preparation of Immunoadhesin

An IL-10-producing melanoma cell strain (JB) and an cell strain not producing IL-10 (ZA) in an amount of 1×10⁵ cells were cultured in complete RPMI medium. The complete RPMI medium was prepared by adding 10% heat-inactivated (deactivated) FCS, 2 mM L-glutamine, nonessential amino acids, 100 IU/ml penicillin and 100 μg/ml streptomycin to RPMI1640.

The melanoma cells were seeded in 0.5 ml of the medium in a 12-well plate 24 hours prior to transfection.

The melanoma cell strains were transfected with pVAX1, and gene expression vectors (pVAX1-IL10R1 (V12: EC*), pVAX1-IL10R1 (EC)/IgG1-Fc (V51: without hinge), pVAX1-IL10R1/IgG1_{—}1-A (V15: SSC type mutated form hinge **) and pVAX1-IL10R1 (EC)/IgG1-Fc (V54: CSC type hinge ***)) prepared in Example 4 or 7, in an amount of 1 μg each.

(2) Measurement of IL-10 Inhibitory Activity

The supernatant was recovered on day 3 following the transfection, and productivity of IL-10 was tested with an ELISA kit (BioSource INTERNATIONAL, Inc., Camarillo, Calif., USA).

75 μl of the medium supernatant of the cells not producing IL-10, and 75 μl of diluted recombinant IL-10 were mixed (final concentrations: 50, 100, 200 and 500 pg/ml), and added to a 96-well plate.

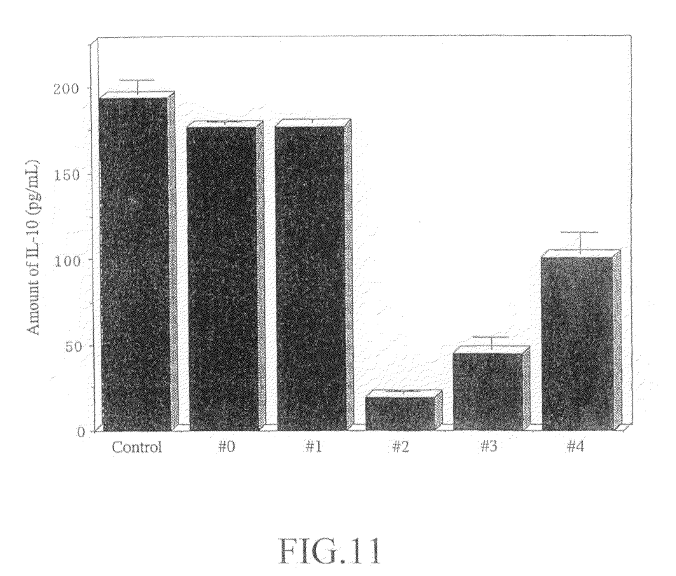

The plate was incubated at 37° for 1 hour. Following the incubation, a substrate was added to each well, and the activity of IL-10 in the supernatant was measured by ELISA. The results are shown in FIG. 11. In FIG. 11, #0 represents pVAX1; #1 represents pVAX1-IL10R1 (V12: EC*); #2 represents pVAX1-IL10R1 (EC)/IgG1 (V51: without hinge); #3 represents pVAX1-IL10R1/IgG1_{—}1-A (V15: SSC type hinge); and #4 represents pVAX1-IL10R1 (EC)/IgG1 (V54: CSC type hinge).

Consequently, pVAX1-IL10R1 (EC)/IgG1 (V51: without hinge), which does not have the hinge part and thus does not form a dimer at the IgG 1 part, inhibited the IL-10 activity best. pVAX1-IL10R1/IgG1_{—}1-A (V15: SSC type hinge) in which dimer formation was inhibited by mutation of cysteine in the hinge part into serine inhibited the IL-10 activity well. Because IL-10 functions as a dimer, it was generally predicted that immunoadhesin which traps IL-10 is also desired to be the form of a dimmer; however, the L-10 inhibitory activity of pVAX1-IL10R1 (EC)/IgG1 (V54: CSC type hinge) was lower than the above two.

All publications, Patents and Patent Applications cited in the present specification are entirely incorporated herein by reference.

### INDUSTRIAL APPLICABILITY

The present invention can be industrially used as antibody drugs, and gene therapeutic drugs.

### Sequence Listing Free Text

SEQ ID NOs: 1 to 6 and SEQ ID NOs: 23 to 29 show primers.

1. A recombinant vector wherein a gene encoding a fusion protein (immunoadhesin), which comprises an extracellular region of an IL-10 receptor bound to a constant region of a human antibody, is incorporated in a vector for gene expression.

2. The recombinant vector according to claim 1 wherein the extracellular region of the IL-10 receptor is the extracellular region of IL-10 receptor 1.

3. The recombinant vector according to claim 1 or 2 wherein the constant region of the human antibody is a region comprising CH2 and CH3 in an Fc region of human IgG1.

4. The recombinant vector according to claim 1 or 2 wherein the constant region of the human antibody comprises CH2 and CH3 in an Fc region of human IgG1, or comprises hinge, and CH2 and CH3 in the Fc region of human IgG1.

5. The recombinant vector according to any one of claims 2 to 4 wherein the extracellular region of the IL-10 receptor 1 is a polypeptide comprising amino acids at position 1-235 or amino acids at position 1-228 in the amino acid sequence of the IL-10 receptor 1 set out in SEQ ID NO: 13.

6. The recombinant vector according to anti one of claims 3 to 5 wherein the Fc region of human IgG1 is a polypeptide encoded by bases from position 70 to 768, bases from position 82 to 768 or bases from position 115 to 768 in the IgG1 gene sequence set out in SEQ ID NO: 12.

7. The recombinant vector according to any one of claims 1 to 6 wherein the expression vector is a plasmid which can replicate in a prokaryote, and which can perform transient expression in mammalian cells.

8. The recombinant vector according to claim 7 wherein the expression vector is pVAX1.

9. The recombinant vector according to claim 2 wherein the fusion protein is a fusion protein that does not form a dimer structure.

10. The recombinant vector according to claim 9 wherein the constant region of the human antibody is the Fc region of human IgG from which the hinge part has been deleted, or within whose hinge part two among the three cysteine residues have been modified into an amino acid residue other than cysteine residue.

11. A recombinant vector which has an IL-10 inhibitory activity, constructed so as to express a fusion protein which comprises an extracellular region of either the IL-10 receptor (a) or (b) below, and a constant region of a human antibody selected from the group consisting of (c), (d) or (e) below:
(a) a polypeptide comprising the amino acids from position 1 to 235 or amino acids from position 1 to 228 in the amino acid sequence of the IL-10 receptor 1 set out in SEQ ID NO: 13;
(b) a peptide having IL-10 receptor activity which is a polypeptide represented by the amino acid sequence 1 to 235 of the IL-10 receptor 1 set out in SEQ ID NO: 13, within which 1 to several amino acids have been deleted, substituted and/or added;
(e) a polypeptide encoded by the gene sequence starting from a base position from 70 to 115 and ending at the base position 768 in the IgG1 gene sequence set out in SEQ ID NO: 12;
(d) a polypeptide having an activity as the Fc region of IgG1, encoded by the gene sequence starting from a base position from 70 to 15 and ending at the base position 768 in the IgG1 gene sequence set out in SEQ ID NO: 12, within which 1 to several amino acids have been deleted, substituted, and/or added;
(e) a polypeptide which is the Fc region of a soluble and mutated form IgG1 that does not form a dimer, encoded by the gene sequence from the base position 70-115 to the base position 768 in the IgG1 gene sequence set out in SEQ ID NO: 12, within which 1 to several amino acids have been deleted, substituted, and/or added.

12. A recombinant vector constructed so as to express a gene encoding a fusion protein which comprises (1) the polypeptide comprising the amino acids from position 1 to 235 or the amino acids from position 1 to 228 in the amino acid sequence of the IL-10 receptor 1 set out in SEQ ID NO: 13, and (2) the polypeptide encoded by the gene of the bases at the positions from 115 to 768, from 82 to 768 or from 70 to 768 in the IgG1 gene sequence set out in SEQ ID NO: 12, in which at least two among the G's at the positions 83, 101 and 110 are substituted with C.

13. A fusion protein wherein an extracellular region of IL-10 receptor 1 is bound to a constant region of a human antibody.

14. The fusion protein according to claim 13 wherein the constant region of the human antibody is (A) a region including CH2 and CH3 in an Fc region of human IgG1, (B) a region comprising CH2 and CH3 in the Fc region of human IgG1, or (C) a region comprising the hinge as well as the CH12 and CH3 in the Fc region of human IgG1.

15. The fusion protein according to claim 13 wherein the fusion protein does not form a dimer structure.

16. The fusion protein according to claim 15 wherein the constant region of the human antibody is the Fc region of human IgG1 from which the hinge part has been deleted, or in whose hinge part at least two among the three cysteine residues has been modified into an amino acid residue other than cysteine residue.

17. A fusion protein having IL-10 inhibitory activity, which comprises an extracellular region of IL-10 receptor 1 selected from the group consisting of:
(a) a polypeptide comprising amino acids at position 1-235 or amino acids at position 1-228 in the amino acid sequence of the IL-10 receptor 1 set out in SEQ ID NO: 13; or
(b) a polypeptide having IL-10 receptor activity, represented by the amino acid sequence 1 to 235 of the IL-10 receptor 1 set out in SEQ ID NO: 13, in which 1 to several amino acids have been deleted, substituted, and/or added, which is bound to an Fc region of IgG1 selected from:
(c) a polypeptide encoded by the gene sequence starting from a base position from 70 to 115 and ending at the base position 768 in the IgG1 gene sequence set out in SEQ ID NO: 12;
(d) a polypeptide having activity as a IgG1-Fc fragment, encoded by the gene sequence starting from a base position from 70 to 115 and ending at the base position 768 in the IgG1 gene sequence set out in SEQ ID NO: 12, in which 1 to several amino acids have been deleted, substituted, and/or added;
(e) a polypeptide which is a soluble mutated IgG1-Fc fragment that does not form a dimmer, encoded by the gene sequence starting from a base position from 70 to 115 and ending at the base position 768 in the IgG1 gene sequence set out in SEQ ID NO: 12, in which 1 to several amino acids have been deleted, substituted, and/or added.

18. A fission protein comprising
(a) a polypeptide comprising the amino acids from position 1 to 235 or the amino acids from position 1 to 228 in the amino acid sequence of the IL-10 receptor 1 set out in SEQ ID NO: 13, and
(b) a polypeptide encoded by the gene of the bases from position 115 to 768 or the bases from position 82 to 768 in the IgG1 gene sequence set out in SEQ ID NO: 12, in which at least two among the G's at the positions 83, 101 and 110 are substituted with C.

19. A gene encoding a fusion protein in which an extracellular region of IL-10 receptor 1 is bound to a constant region of a human antibody.

20. The gene encoding a fusion protein according to claim 19 wherein the constant region of the human antibody is (A) a region including at least the CH2 and CH3 in an Fc region of human IgG1, (B) a region comprising the CH2 and CH3 in the Fc region of human IgG1, or (C) a region comprising the hinge as well as the CH2 and CH3 in the Fc region of human IgG1.

21. The gene according to claim 19 wherein the fusion protein does not form a dimer structure.

22. The gene encoding a fusion protein according to claim 21 wherein the constant region of the human antibody is a modified Fc region of human IgG1 from which the hinge part has been deleted, or in whose hinge part at least two among the three cysteine residues has been modified into an amino acid residue other than cysteine residue.

23. A gene encoding a fusion protein having IL-10 inhibitory activity, which comprises an extracellular region of either the IL-10 receptor (a) or (b) below, and a constant region of a human antibody selected from the group consisting of (c), (d), and (e) below:
(a) a polypeptide comprising the amino acids from position 1 to 235 or the amino acids from position 1 to 228 in the amino acid sequence of the IL-10 receptor 1 set out in SEQ ID NO: 13;
(b) a polypeptide having IL-10 receptor activity, represented by the amino acid sequence from 1 to 235 of the IL-10 receptor 1 set out in SEQ ID NO: 13, in which 1 to several amino acids have been deleted, substituted, and/or added;
(c) a polypeptide encoded by the gene sequence starting from a base position from 70 to 115 and ending at the base position 768 in the IgG1 gene sequence set out in SEQ ID NO: 12;
(d) a polypeptide having an activity as the Fc region of IgG1, encoded by the gene sequence starting from a base position from 70 to 115 and ending at the base position 768 in the IgG1 gene sequence set out in SEQ ID NO: 12, in which 1 to several amino acids have been deleted, substituted, and/or added;
(e) a polypeptide which is the Fc region of a soluble mutated form IgG I that does not form a dimmer, encoded by a gene sequence starting from a base position from 70 to 115 and ending at the base position 768 in the IgG1 gene sequence set out in SEQ ID NO: 12, in which 1 to several amino acids have been deleted, substituted, and/or added.

24. A gene encoding a fusion protein which comprises (1) a polypeptide comprising the amino acids from position 1 to 235 or the amino acids from position 1 to 228 in the amino acid sequence of the IL-10 receptor 1 set out in SEQ ID NO: 13, and (2) a polypeptide encoded by the genes of the bases from position 115 to 768 or the bases from position 70 to 768 in the IgG1 gene sequence set out in SEQ ID NO: 12, in which at least two among the G's at the positions 83, 101 and 110 are substituted with C.

25. A host wherein a recombinant vector that expresses the gene according to any one of claims 19 to 24 is introduced.

26. A process for producing a fusion protein in which an extracellular region of IL-10 receptor 1 is bound to a constant region of a human antibody, the process comprising using the host according to claim 25.

27. A fusion protein produced by the process according to claim 26.

28. A fusion protein obtained by incorporating into pVAX:
(1) a gene encoding an IL-10R1 region, which can be obtained by performing RT-PCR using a primer #1(GCCCCCAAGCTTGCCGCCACCATGCTGCCGTGCCTCG) (SEQ ID NO: 1) and a primer #3 (ATCGGGGGATCCGTTGGTCACGGTGAAATACTGC) (SEQ ID NO: 2) on total RNA collected from Human T-Cell Leukemia (Jurkat), and
(2) (i) a gene encoding an Fc region of IgG1 from which a hinge has been deleted, which can be obtained by performing PCR using #4 (CGCGGATCCGCACCTGAACTCCTGGG) (SEQ ID NO: 5) as a forward primer and #7 (ATCGGGGAATTCTCATTTACCCGGAGACAGGG) (SEQ ID NO: 6) as a reverse primer for excision of IgG-Fc_{—}1 (region 1: without hinge part) on SRα-neo1-CD80/CD86/IgFc,
(ii) a gene encoding an Fc region of IgG1 having a mutated form hinge which can be obtained by performing PCR using #5 (CGGGATCCTCTGACAAAACTCACACATCC) (SEQ ID NO: 4) as a forward primer and #7 (ATCGGGGAATTCTCATTTACCCGGAGACAGGG) (SEQ ID NO: 6) as a reverse primer for excision of IgG1-Fc2 (region 2: mutated form hinge part) from SRα-neo1-CD80/CD86/IgFc, and further modifying the thus resulting gene using #8 (CTCACACATCCCCACCGTCCCCAGCACCTG) (SEQ ID NO: 25) as a forward primer and #9 (ACGGTGGGGATGTGTGAGTTTTGTCAGAAGA) (SEQ ID NO: 26) as a reverse primer, or
(iii) a gene encoding an Fc region of IgG1 having a wild type hinge which can be obtained by performing PCR using #6: IgG1_{—}2_F_BamH (CGGGATCCTCTGACAAAACTCACACATCC) (SEQ ID NO: 4) as a forward primer and #7 (ATCGGGGAATTCTCATTTACCCGGAGACAGGG) (SEQ ID NO:6) as a reverse primer for excision of IgG1-Fc_{—}2 (region 3: wild type hinge part) from SRα-neo1-CD80/CD86/IgFc, and further modifying the thus resulting gene using #10 (GTGACAAAACTCACACATGCCCACCGTGCC) (SEQ ID NO: 28) as a forward primer and #11 (ATGTGTGAGTTTTGTCACAAGATTTGGACTC) (SEQ ID NO: 29) as a reverse primer for modification, and expressing this gene.
